(19) United States
(12) Patent Application Publication
(10) Pub. No.: US 2022/0354566 A1
(43) Pub. Date: Nov. 10, 2022

(54) DEVICES, SYSTEMS AND METHODS FOR ASSISTANCE OF BALLOON ABLATION

(71) Applicant: KONINKLIJKE PHILIPS N.V., EINDHOVEN (NL)

(72) Inventors: Yitzhack SCHWARTZ, Caesarea (IL); Zalman IBRAGIMOV, Caesarea (IL); Leonid GLUHOVSKY, Caesarea (IL)

(21) Appl. No.: 17/624,364

(22) PCT Filed: Jun. 30, 2020
(86) PCT No.: PCT/EP2020/068334

§ 371 Date: Jan. 3, 2022

(30) Foreign Application Priority Data
Jul. 3, 2019 (EP) 19184189.9
Jun. 11, 2020 (EP) 20179587.9

(51) Int. Cl. A61B 18/14; A61B 34/10; A61B 34/00; A61B 5/06; A61B 34/20

(52) U.S. Cl. CPC A61B 18/1492; A61B 34/10; A61B 34/25; A61B 5/063; A61B 5/068; A61B 34/20; A61B 2018/00577

(57) ABSTRACT

Systems, devices, and methods for guiding an ablation procedure are provided. For example, in one embodiment, a system for guiding ablation includes a processor circuit in communication an electrophysiology (EP) catheter comprising a plurality of electrodes. The EP catheter is positioned near an ablation balloon during placement at the ablation site, and is used to detect blood flow within a cavity of the heart by detecting electrical signals relating to dielectric properties. It can then be determined whether any gaps are present at the interface between the balloon and the ablation site. For example, the processor circuit can determine, based on the detected blood flow, whether a balloon occludes a region of interest. The processor then outputs a visualization indicating whether the balloon occludes the region of interest to a display.

### TECHNICAL FIELD OF THE INVENTION

The present disclosure relates generally to electrophysiological imaging for assisting or guiding treatment procedures, and, in particular, to electrophysiological systems and methods for imaging volumes of the body and assisting or guiding balloon ablation procedures.

### BACKGROUND OF THE INVENTION

Atrial fibrillation (AF) is an abnormal heart rhythm characterized by rapid and irregular beating of the atria, and may be associated with heart palpitations, fainting, lightheadedness, shortness of breath, or chest pain. The disease is associated with an increased risk of heart failure, dementia, and stroke. AF may be caused by electrical pulses generated by secondary pacers at the ostium of the pulmonary veins. Accordingly, one way of treating AF is by pulmonary vein isolation, which can include ablating the inner wall of the left atrium to form lesions that isolate the ostium of the pulmonary veins from the rest of the left atrium. Ablation can be performed in various ways, including radiofrequency (RF) ablation, ultrasonic ablation, and cryoablation. RF ablation is a conventional ablation procedure that involves powering an RF electrode to create contiguous, transmural lesions using heat energy. RF ablation suffers from some drawbacks, such as a longer procedure time and small gaps in the lesions that cause AF to return over time, and even immediately.

Balloon-based ablation procedures, such as cryoablation, are alternative AF treatment procedures that are advantageous in some respects, including a shorter procedure time and the ability to create a contiguous lesion around the pulmonary vein ostium in a single shot. In cryoablation, an inflatable cryoballoon is inflated and cooled to a temperature (e.g., below −65° C.) that causes an electrically-isolating lesion or firewall in the tissue. In conventional systems, the cryoballoon is guided to the ablation site, typically the ostium of a pulmonary vein, using fluoroscopy. As shown in FIGS. 1A and 1B, the cryoballoon 30 is inflated, cooled, and positioned to fully occlude blood flow of the pulmonary vein 10 from the left atrium 20. In this way, it can be assured that the lesion formed from the cryoablation will electrically isolate the pulmonary vein from the left atrium.

Some challenges with balloon ablation include guiding the ablation balloon (e.g., cryoballoon) to the ablation site, and ensuring that the balloon is placed and oriented to maintain contact with a full circumference of the ostium. If the balloon is misaligned during ablation, the resulting lesion can include one or more gaps which may result in reconduction and recurrence of the arrhythmia thereby requiring a re-do procedure when the AF symptoms return. Referring again to FIG. 1A, in conventional methods, angiographic and/or fluoroscopic procedures are used to guide the balloon to the ablation site. Once the inflated balloon 30 is in place, a Fluoroscopic contrast agent 12 also referred to as dye herein is introduced into the pulmonary vein 10 to ascertain pulmonary vein occlusion and detect residual leaks or gaps in the interface between the balloon 30 and the ostium of the pulmonary vein 10 prior to ablating the tissue. When gaps exist, a portion 14 of dye will leak through into the left atrium 20. This leaked portion 14 can be detected by angiography and in particular venography under fluoroscopy, which indicates that the balloon is not yet fully occluding blood flow from the pulmonary vein into the left atrium, and therefore is not optimally positioned to completely isolate the pulmonary vein from the left atrium. Accordingly, the physician can adjust and reposition the balloon until no residual leaks of the dye are seen, as shown in FIG. 1B.

There are some shortcomings to using X ray based angiography or venography. to guide balloon ablation procedures. For example, patients and physicians may prefer to avoid x-ray radiation emitted during such procedures. Furthermore, guiding the balloon to the ablation site and checking for leaks in the pulmonary vein-balloon interface may be an imprecise and difficult processes that requires special expertise. It may be contraindicated or unadvisable to use dye for some patients. For example, at least 20% of the population has some type of contraindication for usage of dye, including allergic reactions and kidney failure. Furthermore, due to inherent limitations of dye injection under 2D fluoroscopy, approximately 13% of residual leaks may not be apparent on venography.

There are two additional methods that can support and further assist in verifying PV occlusion and establishing optimal cryoballoon appositioning:

(i) Continuous pressure monitoring identifies PV occlusion during sinus rhythm by a loss of the A wave and a change in the amplitude (increase) and morphology of the V wave. During AF, PV occlusion is identified by an abrupt increase in the V wave amplitude with a loss of the small continuous atrial A waves;

(ii) Intra-Cardiac Echocardiography (ICE) visualizes the cryoballoon in 2D or 3D and identifies leaks by presence of “micro-bubbles” (agitated saline) as well as color Doppler flow jets.

WO 2013/022853 and US 2017/347896 each disclose balloon ablation systems and methods, in which impedance sensing is used to distinguish between conduction paths through tissue and conduction paths through blood, in order to determine if the balloon has provided the desired occlusion. WO2018/207128 discloses balloon ablation system using catheter electrodes to image the heart and also to provide leakage detection.

### SUMMARY OF THE INVENTION

The invention is defined by the claims and they define devices, systems methods, computer program products and computer readable media comprising the computer program products all for assisting (for example guiding) a balloon ablation therapy procedure using an ablation balloon for occluding a cavity of a subject during the procedure.

All of these aspects relate to the use of data that represent one or more electrical signals measured using one or more of a plurality of electrodes disposed on an elongate tip member (122) of an electrophysiology catheter (120) when one or more of the plurality of electrodes are positioned distally of the ablation balloon (30) in the anatomical cavity wherein the electrical signals are responsive to local dielectric properties within the anatomical cavity and were measured responsive to injection of a dielectric medium into the anatomical cavity. The data may be processed to identify a change of at least one characteristic of the one or more electrical signals where the at least one change is responsive to the injection of the dielectric medium; to determine from the identified at least one change occlusion information relating to the occlusion of the anatomical cavity by the ablation balloon. Optionally the output data is generated comprising the occlusion information. The output data may be provided to a user such as a physician, surgeon, caregiver or even the subject such as for example a caregiver performing the procedure.

The occlusion information may assist the user in performing the procedure as the occlusion information may be of relevance to and/or of influence on for example the course and/or outcome of the procedure. For example, occlusion information indicating full occlusion during the procedure may prompt a user to start an ablation. Alternatively, occlusion information indicating incomplete occlusion may be used by the user to try and improve occlusion for example to achieve full occlusion. The occlusion information may guide the user in performing the procedure.

It was found that injection of a dielectric medium with dielectric properties different from those of blood may alter the local dielectric properties in the vicinity of the point of injection of such dielectric medium in a blood containing anatomical cavity. The electrodes of the EP catheter can be used to register changes of the local dielectric properties due to the injection by measuring electrical signals, such as for example voltages and/or currents, on the electrodes of the EP catheter before during and after injection of the medium when the electrodes are positioned in the blood pool of the anatomical cavity distally of the balloon used during the ablation procedure. By identifying the changes in the signals occlusion information can be deduced.

The data processing allows the use of a dielectric medium and this may improve the detection of small leaks during occlusion with the procedure.

Being able to generate or having the occlusion information may obviate the use of X-ray based contrast agent dependent fluoroscopy, angiography or venography which currently are used to check for small leaks. The current aspects allow a dielectric medium (dielectric contrast agent) based detection of small leaks during an occlusion and such dielectric medium need not be X-ray absorbing or emitting. Note however, that in the current aspects an X-ray absorbing contrast agent (as for example known in the art) may still be used as long as it can serve as a dielectric medium in the context of the current disclosure.

In some embodiments or examples of the proposed aspects the at least one change of characteristic comprises one or more of: the amplitude of a signal spike; the time decay of a signal spike; and a comparison of the signal amplitude before and after a signal spike.

In some embodiments or examples of the proposed aspects the data represent a plurality of electrical signals each one measured using one of the plurality of electrodes and wherein the data processor is configured to identify the at least one change of the characteristic for each of the plurality of signals.

In some embodiments or examples of the proposed aspects, for example by configuring the processor circuit or data processor to this end, based on the change of the at least one characteristics, changes in local dielectric properties within the cavity are determined.

In some embodiments or examples of the proposed aspects the electrical signals further comprise baseline electrical signals measured when the anatomical cavity is not occluded by the ablation balloon. In such case, for example by configuring the processor circuit and/or the data processor to this end, a degree of occlusion is determined using a model based on a comparison of the baseline signal to a signal obtained after or during occlusion. In some of these embodiments or examples, for example by configuring the processor circuit and/or the data processor to this end, an amount of the identified change of the baseline signal before occlusion and after occlusion is compared to a threshold and when the amount of change exceeds the threshold a regional occlusion is determined to exist.

In some embodiments or examples of the proposed aspects, for example by configuring the processor circuit and/or the data processor to this end, shadow position indication of the electrophysiology catheter in the vicinity of the location at which the occlusion determination is to be obtained is used to determine therefrom one or more of: the distance from the elongate tip member to the mapping data; the distance from the elongate tip member to the ostium of a pulmonary vein of the region interest; and the current distance from the elongate tip member to the shadow position.

In some embodiments or examples of the proposed aspects, for example by configuring the processor circuit and/or the data processor to be able to communicate with the one or more of a plurality of electrodes disposed on the elongate tip member (122) of the electrophysiology catheter (120), the one or more of the plurality of electrodes are controlled measure the electrical signals. This may entail controlling these electrodes to provide electrical signals in order to invoke or cause the electrical signals to be measured.

In some embodiments or examples of the proposed aspects, for example by configuring the processor circuit and/or the data processor to this end, the occlusion information is generated to comprise a visualization of at least part to of the occlusion information indicating whether the ablation balloon at least partially occludes the anatomical cavity corresponding to, near, or at a location of each of the one or more of the plurality of electrodes.

In some embodiments or examples of the proposed aspects, for example by configuring the processor circuit and/or the data processor to this end, data representative of a map of at least part of the anatomical cavity relevant to the procedure is used to generate the map, a visualization of the occlusion information is generated and output data comprising the map and the visualization is generated. The visualization on the map may be generated as an overlay. The visualization can indicate a location of a gap between the ablation balloon and the anatomical cavity.

In some embodiments or examples of the proposed aspects, for example by configuring the processor circuit and/or the data processor to communicate with the one or more of the plurality of electrodes and with a plurality of external body patch electrodes for positioning on a subject to apply an electrical field to at least the anatomical cavity using body patch electrical signals, the external body patch electrodes are controlled to provide the electrical field; the one or more of the plurality of electrodes are controlled to detect distortions in the generated electrical field and the map data are generated to comprise the detected distortions; and, based on the detected distortions, a map of the anatomical cavity relevant to the procedure is generated.

In embodiments the processor circuit is configured to control each of the one or more electrodes of the plurality of electrodes to emit a respective electrical signal at a different frequency; and receive the respective electrical signals from other electrodes of the one or more of the plurality of electrodes.

In embodiments, the processor circuit is configured to: receive, via an input, lesion validation data of a region of a wall of the anatomical cavity; determine, based on the lesion validation data, whether any gaps exist in a lesion created by the ablation balloon.

In embodiments, the processor circuit is configured to communicate with the one or more of the plurality of electrodes and to control the plurality of electrodes to obtain lesion validation data of a region of the wall of the anatomical cavity; determine, based on the lesion validation data, whether any gaps exist in a lesion created by the ablation balloon.

In such embodiments output data may be generated that comprise an indication of whether the gaps exist in a lesion and such lesion output data may be provided to a user interface for providing the indication to a user.

According to an aspect there is proposed a system comprises a device as claimed in any of the previous claims wherein the processor circuit comprises an output, communicatively coupled to the data processor; and a user interface communicatively coupled to the processor circuit at least via the output and configured to provide an indication of the occlusion information to a user.

In some embodiments the system comprises a controller for communicatively coupling to the one or more of the plurality of electrodes and to the plurality of body patch electrodes, and communicatively coupled to the processing circuit, the controller being configured to supply any electrical signals for any electrodes upon control of any of the electrodes by the processing circuit;

In some embodiments the system comprises an injection system for injecting the medium, and optionally a vessel comprising the medium.

In some embodiments the system comprises the ablation balloon (30); and the electrophysiology catheter (120). The ablation balloon preferably is part of a balloon ablation catheter comprising the electrophysiology catheter.

In another aspect there is proposed a method for assisting a balloon ablation therapy procedure using an electrophysiology catheter and ablation balloon for occluding a cavity of a subject during the procedure during which procedure data are generated that represent one or more electrical signals measured using one or more of a plurality of electrodes disposed on an elongate tip member (122) of the electrophysiology catheter (120) when one or more of the plurality of electrodes are positioned distally of the ablation balloon (30) in the anatomical cavity and wherein the electrical signals are responsive to local dielectric properties within the anatomical cavity and were measured responsive to injection of a dielectric medium into the anatomical cavity, the method comprising: receiving, at an input of a processor circuit, the data; processing, by a data processor communicatively coupled to the input, the received data and identify a change of at least one characteristic of the one or more electrical signals where the at least one change is responsive to the injection of the dielectric medium; determining, by the data processor, from the identified at least one change occlusion information relating to the occlusion of the anatomical cavity by the ablation balloon; and optionally, provide, using a user interface communicatively coupled to an output of the processor circuit and communicatively coupled to the data processor, the occlusion information to a user.

There are proposed a computer program products comprising instructions for implementing any method as defined herein when the it is run on a processor circuit and/or data processor of the device as defined herein.

There are proposed computer readable media comprising the computer program products as defined herein.

Embodiments of the methods, computer program products and the computer readable media including the same have been described herein above. Thus any steps and features defined for the device and/or system may be used to define steps and features of the method, computer program product and media comprising such computer program product.

Other examples and embodiments may be defined as follows.
A balloon ablation system, comprising:

an ablation balloon;

an electrophysiology catheter comprising an elongate tip member configured to be positioned distally of the ablation balloon;

a plurality of electrodes positioned on the elongate tip member; and

an apparatus for guiding ablation within a cavity of a heart, comprising a processor circuit in communication with the electrophysiology catheter, wherein the processor circuit is configured to:
control the plurality of electrodes to emit and detect a plurality of electrical signals;
detect, based on the detected plurality of electrical signals, changes in local dielectric properties within the cavity of the heart distally of the ablation balloon;
determine, based on the detected local dielectric properties, information relating to the occlusion by the ablation balloon of a region of interest, based on the response of one or more of the detected plurality of electrical signals to injection of a medium; and
output, to a display in communication with the processor circuit, a first visualization indicating said information relating to the occlusion by the ablation balloon of the region of interest.

Aspects of the present disclosure thus provide systems, devices, and methods for guiding an ablation procedure. For example, in one embodiment, an apparatus includes a processor circuit configured to control an electrophysiology (EP) catheter to emit and detect electrical signals indicating blood flow near the electrodes. The EP catheter can be positioned near an ablation balloon during placement at the pulmonary vein (PV) ostium, and prior to ablation. Based on the blood flow (or lack thereof) detected by the EP catheter based on analysis of dielectric properties, the apparatus can determine whether any gaps are present at the interface between the balloon and the PV ostium. Further, in some embodiments, the apparatus can determine the location of the gaps to aid a physician in repositioning the balloon. Accordingly, the ablation procedure may be advantageously guided by the apparatus without the use of fluoroscopy, angiography, and/or contrast agent.

In some embodiments, the processor circuit is configured to determine a degree of occlusion of the balloon on the region of interest. In some embodiments, the processor circuit is configured to determine the degree of occlusion using a model based on a comparison of a pre-occlusion baseline signal to a signal obtained after occlusion of the region of interest. In some embodiments, the processor circuit is configured to compare an amount of change between the pre-occlusion baseline signal and the signal obtained after occlusion to a threshold, and the processor circuit is configured to determine regional occlusion when the amount of change exceeds the threshold. In some embodiments, the processor circuit is configured to control the plurality of electrodes to: obtain mapping data of the cavity of the heart; generate, based on the mapping data obtained by the electrode assembly, a map of the cavity of the heart, wherein the map includes a region of interest for ablation; and output, to a display in communication with the processor circuit, the map of the cavity of the heart and a second visualization of a position of the balloon within the cavity in the map.

In some embodiments, the processor circuit is in communication with a plurality of external body patch electrodes configured to be positioned on a body of a patient. In some embodiments, the processor circuit is configured to: control the external body patch electrodes to emit electrical fields into the cavity of the heart; control the plurality of electrodes to detect distortions in the electrical fields; and generate the mapping data based on the detected distortions in the electrical fields. In some embodiments, the processor circuit is configured to overlay the second visualization on the map of the cavity of the heart at the region of interest, wherein the second visualization indicates a location of a leak between the balloon and the region of interest. In some embodiments, the processor circuit is configured to: control the plurality of electrodes to obtain lesion validation data of the region of interest; determine, based on the lesion validation data, whether any gaps exist in a lesion created by the balloon; and output, to the display, a third visualization indicating whether gaps exist in the lesion. In some embodiments, the processor circuit is configured to overlay the third visualization on the map of the cavity of the heart at the region of interest, wherein the third visualization indicates a location of a gap in the lesion. In some embodiments, the processor circuit is configured to control each of the plurality of electrodes to: emit a respective electrical signal at a different frequency; and receive the respective electrical signals from other electrodes of the plurality of electrodes. In some embodiments, a system includes the processor circuit described herein and the electrophysiology catheter. The electrophysiology catheter may include an elongate tip member configured to be positioned distally of the balloon, wherein the plurality of electrodes is positioned on the elongate tip member.

The invention also provides a method for guiding ablation within a cavity of a heart, comprising:

controlling, by a processor circuit, a plurality of electrodes of an electrophysiology catheter to emit and detect a plurality of electrical signals, wherein the plurality of electrodes are formed on an elongate tip member of an electrophysiology catheter positioned distally of an ablation balloon;

detecting, based on the detected plurality of electrical signals, changes in local dielectric properties within the cavity of the heart distally of the ablation balloon;

determining, based on the changes in dielectric properties, information relating to the occlusion by the ablation balloon of a region of interest, based on the response of one or more of the detected plurality of electrical signals to injection of a medium; and

outputting, to a display in communication with the processor circuit, a first visualization indicating said information relating to the occlusion by the ablation of the region of interest.

In some embodiments, the method further comprises controlling, by the processor circuit, the plurality of electrodes to: obtain mapping data of the cavity of the heart; and generate, based on the mapping data obtained by the electrode assembly, a map of the cavity of the heart, wherein the map includes a region of interest for ablation. The method may further comprise outputting, to a display in communication with the processor circuit, the map of the cavity of the heart and a second visualization of a position of the balloon within the cavity in the map. In some embodiments, the method further includes controlling, by the processor circuit, a plurality of external body patch electrodes positioned on a body of the patient to emit electrical fields into the cavity of the heart; controlling the plurality of electrodes to detect distortions in the electrical fields; and generating the mapping data based on the detected distortions in the electrical fields.

In some embodiments, the method further includes overlaying the second visualization on the map of the cavity of the heart at the region of interest, wherein the second visualization indicates, on the map, a location of a leak between the balloon and the region of interest. In some embodiments, the method further comprises: controlling the plurality of electrodes to obtain lesion validation data of the region of interest; determining, based on the lesion validation data, whether any gaps exist in a lesion created by the balloon; and outputting, to the display, a third visualization indicating whether gaps exist in the lesion.

In some embodiments, the method further comprises overlaying the third visualization on the map of the cavity of the heart at the region of interest, wherein the third visualization indicates a location of a gap in the lesion. In some embodiments, the method further comprises controlling each of the plurality of electrodes to: emit a respective electrical signal at a different frequency; and receive the respective electrical signals from other electrodes of the plurality of electrodes.

According to another embodiment of the present disclosure, a computer program product includes a non-transitory computer-readable medium having program code recorded thereon. The program code includes: code for causing a processor circuit to control a plurality of electrodes of an electrophysiology catheter to emit and detect a plurality of electrical signals; code for causing the processor circuit to detect, based on the detected plurality of electrical signals, blood flow within the cavity of the heart; code for causing the processor circuit to determine, based on the detected blood flow, whether a balloon at least partially occludes a region of interest; and code for causing the processor circuit to output, to a display in communication with the processor circuit, a visualization indicating whether the balloon at least partially occludes the region of interest.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1A is a graphical depiction of a cryoballoon positioned at the PV ostium with incomplete occlusion.

FIG. 1B is a graphical depiction of a cryoballoon positioned at the PV ostium with complete occlusion.

FIG. 2 is a diagrammatic view of an EP-guided ablation system, according to aspects of the present disclosure.

FIG. 3 is a diagrammatic view of a processor circuit, according to aspects of the present disclosure.

FIGS. 4A to 4C are flow diagrams of methods for performing an EP-guided balloon ablation procedure, according to aspects of the present disclosure.

FIG. 5 is a graphical view of a user interface for EP-guided balloon ablation, according to aspects of the present disclosure.

FIG. 6A is a graphical depiction of a cryoballoon and EP catheter positioned at the PV ostium with complete occlusion, according to aspects of the present disclosure.

FIG. 6B is a graphical depiction of an RF ablation balloon and EP catheter positioned at the PV ostium with complete occlusion, according to aspects of the present disclosure.

FIG. 7 is a flow diagram of a method for determining PV occlusion by an ablation balloon, according to aspects of the present disclosure.

FIG. 8A is a graph of an electrical signal detected by a first electrode of an EP catheter during an incomplete occlusion, wherein the first electrode is spaced from a leak between a balloon and an ablation site, according to aspects of the present disclosure.

FIG. 8B is a graph of an electrical signal detected by a second electrode of an EP catheter during the incomplete occlusion, according to aspects of the present disclosure, wherein the second electrode is adjacent to a leak between the balloon and the ablation site, according to aspects of the present disclosure.

FIG. 9 is a diagrammatic view of a natives matrix of electrical signals used to determine PV occlusion by a balloon, according to aspects of the present disclosure.

FIGS. 10A and 10B show internal electrode signals when there is full occlusion.

FIGS. 11A and 11B show internal electrode signals when there is a large residual leak.

FIGS. 12A and 12B show internal electrode signals when there is a moderate residual leak.

FIGS. 13A and 13B show internal electrode signals when there is a small residual leak.

FIG. 14 is a graphical view of a user interface for EP-guided balloon ablation, according to aspects of the present disclosure.

FIG. 15 is a diagrammatic view of a post-ablation validation procedure, according to aspects of the present disclosure.

FIG. 16 is a graphical view of a user interface for validating an ablation lesion, according to aspects of the present disclosure.

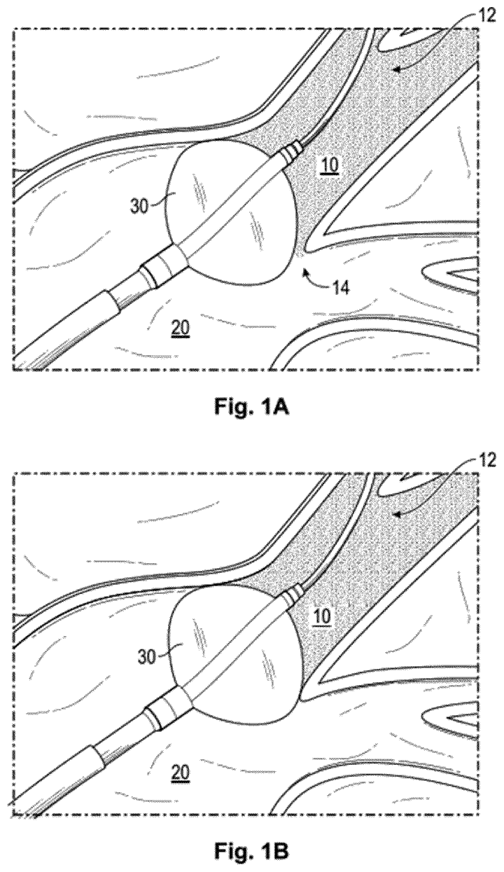

### DETAILED DESCRIPTION OF THE EMBODIMENTS

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. For example, although the following disclosure may refer to embodiments that include cryoablation procedures, cryoballoons, cryocatheters, RF balloon ablation, or RF balloons, it will be understood that such embodiments are exemplary, and are not intended to limit the scope of the disclosure to those applications. For example, it will be understood that the devices, systems, and methods described herein are applicable to a variety of treatment procedures in which a balloon is used to occlude a body lumen or body cavity. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

As mentioned above, fluoroscopy-based approaches used in guiding balloon ablation procedures, including navigation and deployment of the balloon at the PV ostium and detecting leaks between the PV ostium and the ablation balloon suffer from various drawbacks. It may be desirable to provide an approach for guiding a full ablation procedure without using fluoroscopy and/or contrast agent. The present disclosure provides systems, methods, and devices for guiding a balloon ablation procedure using EP imaging and EP-based leak detection techniques. As will be further described below, an electrophysiology system can be used to provide for image-guided delivery of a balloon ablation device, such as a cryoballoon or RF balloon, into a body cavity, such as the left atrium, and to facilitate a fully-occluding placement of the cryoballoon at an ablation site.

A region of interest of a subject can comprise an anatomical cavity of the subject such as a chamber of a heart and or a part of a vessel (artery or vein) connected to such chamber.

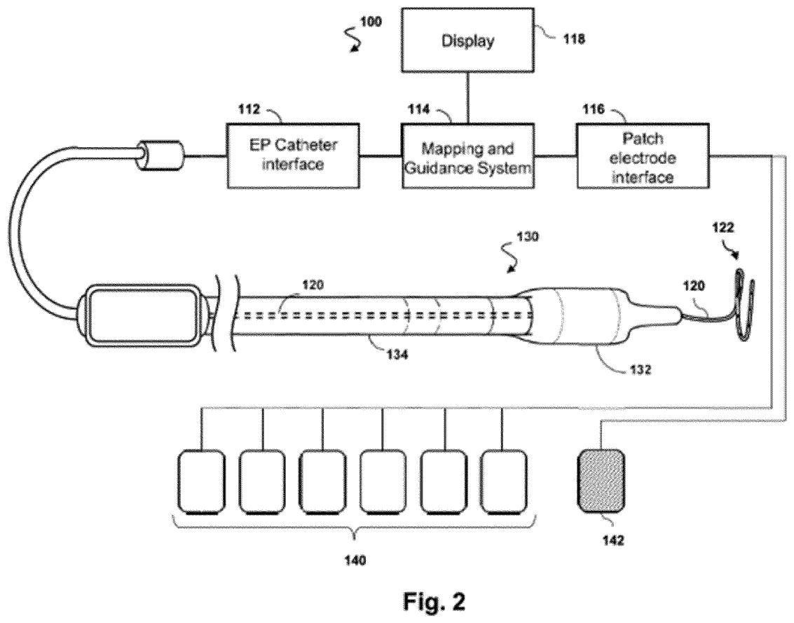

FIG. 2 is a diagrammatic schematic view of an EP cryoablation system 100, according to aspects of the present disclosure. The EP cryoablation system 100 includes an EP catheter 120. In some embodiments, the EP catheter 120 extends through a lumen of a cryoballoon catheter 130. The EP catheter 120 is communicatively coupled to an EP catheter interface 112, which is communicatively coupled to a mapping and guidance system 114. The cryoballoon catheter 130 may include an inflatable cryoballoon 132 coupled to a distal portion of a flexible elongate member 134, which may comprise a sheath. The EP catheter 120 may be positioned at least partially within a lumen of the flexible elongate member 134 such that a distal portion 122 of the EP catheter 120, which comprises a plurality of electrodes positioned on an elongate tip member, may protrude out a distal end of the cryoballoon catheter 130. For example, the cryoballoon catheter 130 may comprise a lumen configured to slidably receive the EP catheter 120. The EP catheter 120 may comprise a flexible elongate member configured to be positioned within the cryoballoon catheter 130. In some embodiments, the cryoballoon catheter 130 may be introduced into the body cavity first and the EP catheter 120 may be advanced distally within the cryoballoon catheter 130 until the distal portion 122 of the EP catheter 120 protrudes out the distal end of the cryoballoon catheter 130 at a region of interest (e.g., an ablation site).

The distal portion 122 of the EP catheter may comprise a plurality of electrodes positioned on the elongate tip member. In some embodiments, the EP catheter comprises between 8 and 10 electrodes. However, the EP catheter can include other numbers of electrodes, including 2, 4, 6, 14, 20, 30, 60, or any other suitable number of electrodes, both larger and smaller. The elongate tip member may be configured to be positioned distally of the cryoballoon, and may be biased, shaped, or otherwise structurally configured to assume a shape, such as a circular shape, in which the electrodes are spaced from one another about one or more planes. For example, the EP catheter can be a spiral mapping catheter (SMC) in which electrodes are distributed along an elongate tip member in a spiral configuration, e.g. with a 15 mm, 20 mm or 25 mm loop diameter. In some embodiments, commercially-available EP catheters can be used with the system 100, including the Achieve™ and Achieve Advance™ Mapping catheters manufactured by Medtronic™. The EP catheter can be designed for use with the Arctic Front™ Family of Cardiac Cryoablation Catheters and/or the FlexCath™ Advance Steerable Sheath, manufactured by Medtronic™. In some embodiments, the cryoballoon 132 comprises a plurality of electrodes positioned on an exterior surface of the cryoballoon 132 and configured to obtain data used to determine occlusion at an ablation site. Further details regarding EP catheters and assemblies can be found in, for example, U.S. Pat. No. 6,002,955, titled “Stabilized Electrophysiology Catheter and Method for Use,” the entirety of which is hereby incorporated by reference.

The system 100 further comprises a plurality of body patch electrodes 140 and a reference patch electrode 142 communicatively coupled to a patch electrode interface 116, which is in communication with the mapping and guidance system 114. For example, the patch electrodes 140 and the reference electrode 142 may be coupled to the patch electrode interface 116 via electrical cables. In the embodiment shown in FIG. 2, the system 100 comprises six external body patch electrodes 140 and one reference electrode 142. However, in some embodiments, the system 100 comprises fewer or more body patch electrodes 140 than are shown in FIG. 2, including 1, 2, 4, 8, 19, 12, 20, or any other suitable number of body patch electrodes, both lager and smaller. Further, in some embodiments, the system 100 may include more than one reference electrode 142, including 2, 4, 5, or any other suitable number of reference patch electrodes. The patch electrodes 140 and reference electrode 142 may be used in generating images or models of a body cavity, such as the chambers of the heart, body lumens that provide access to the body cavity, and other features identified to be electrically isolated from the body cavity (e.g., the pulmonary vein). For example, the patch electrodes 140 may be used in pairs to generate electrical fields in different directions and at different frequencies within a body cavity of a patient. The patch electrodes 140 may be controlled by the mapping and guidance system 114 and/or the patch electrode interface 116 to generate the electrical fields. The patch electrode interface and or the catheter interface may be part of a controller. Mapping data is obtained by the electrodes of the EP catheter 120 by detecting distortions in the electrical fields. The mapping data may then be used to generate the image or model of the body volume, such as a cavity of the heart. The mapping and guidance system 114 may then output the generated map of the cavity to a display. Further, the mapping and guidance system 114 may be configured to determine a location of the EP catheter within the body cavity and output a visualization to the display that indicates a position of the EP catheter 120 within the map. For example, based on the mapping data acquired by the electrodes of the EP catheter, the mapping and guidance system 114 may be configured to determine the position of the electrodes of the EP catheter, and output a visualization indicating the position of each of the electrodes on the map (see FIG. 5, indicator 312). Further, based on the determined position of the electrodes of the EP catheter, the mapping and guidance system 114 may be configured to determine or estimate a location of the cryoballoon 132 within the body cavity. For example, in some embodiments, the EP catheter 120 is first advanced to a region of interest, such as the ostium of a pulmonary vein, and anchored into place. The cryoballoon catheter 130 may then be advanced over the EP catheter to the region of interest. If the distance and position of the cryoballoon 132 relative to the electrodes of the EP catheter 120 are fixed and known, the mapping and guidance system 114 may be configured to identify or infer a location of the cryoballoon 132 by a translation of the coordinates of the EP catheter 120. For example, the distance between the cryoballoon 132 and the electrodes of the EP catheter 120 may be added/subtracted to the coordinates of the EP catheter to identify the location of the cryoballoon 132. Accordingly, the mapping and guidance system 114 may be configured to output a visualization of the position of the cryoballoon 132 with respect to the map of the body cavity.

A more detailed explanation of the use of body patch electrodes and catheter electrodes to map body volumes and visualize the locations of EP catheters within the map can be found in, for example, U.S. Pat. No. 10,278,616, titled “Systems and Methods for Tracking an Intrabody Catheter,” and U.S. Pat. No. 5,983,126, titled “Catheter Location System and Method,” the entireties of which are hereby incorporated by reference as well as in publications WO2018130974 and WO2019034944 of the corresponding international patent applications the entireties of which are herein incorporated by reference.

For completeness, an outline will be presented of the imaging function. The electrodes of the EP catheter may be labelled “internal electrodes” and the patch electrodes may be labelled “external electrodes”. A distance between each of the internal electrodes (inter-electrode spacing) and their corresponding electrical weight lengths are predetermined and hence known.

The mapping guidance system 114 is configured to provide and receive signals from the electrodes to perform a cardiac dielectric imaging process. It provides 3D electro-anatomical visualization for guiding catheter-based treatment of cardiac arrhythmias. It utilizes wide-band dielectric sensing and a technology based on the bending of electric fields to generate high-resolution ‘CT-like’ full 3D images as well as flattened 3D panoramic views of the cardiac anatomy. Sensor-less diagnostic and ablation EP catheters are prequalified to operate with the system.

The system generates a global low intra-body electrical field by the set of external sensors, which are differentially excited, together with a local electrical field via the internal electrodes on the indwelling catheter. The internal and external electrodes are all both emitters and receivers in the frequency range of 20 kHz-100 kHz. A right leg sensor serves as an electric reference for all voltage measurements (creating a V-space).

The distribution of the induced electric field is inherently inhomogeneous due to the different dielectric properties and absorption rates (related to conductivity) of the interrogated tissues. The external electrodes measure the global general effects and distorted electric field whereas the internal electrodes measure the local effect and tissue response.

Throughout the imaging process, the imaged volume continuously grows at the sampling rate of 100 Hz. An optimal transfer function is used to transform the voltages to Euclidian coordinates (creating an R-space) while maintaining the known pre-qualified catheter characteristics (electrode spacing and electrical weight length, functioning as an internal ruler) as well as a set of other constraints. This transfer function is repeatedly defined and applied globally.

Using the updated R-space cloud of points a reconstruction algorithm generates a 3D image. Regions with inherently marked steep gradients in the electric field (i.e. drainage of vessels into or out of a cardiac chamber as well as the A-V and V-A valves) are picked up uniquely by the system and imaged even without physically visiting them with the catheter. The system thus can image at locations beyond the catheter.

The continuous combined global and local field measurements enable sophisticated continuous detection and effective handling of inconsistencies and outliers, level of electrode coverage (by measuring inter-correlations), pacing (saturation), as well as physiological drift.

Drift is detected by applying a moving window over time and applying continuous correction whereby the catheter location remains accurate throughout the whole procedure making the system resilient to drift. Cardiac and respiratory motion are also compensated.

For imaging, at least two electrodes on a catheter/wire/pacing lead are necessary, whereas even a single electrode can be spatially localized after the initial obligatory computation of the R-field The system has been proposed for guiding CryoBalloon (CB) Pulmonary Vein Isolation (PVI) procedures while potentially minimizing exposure to fluoroscopy and obviating dye injection.

In the diagram shown in FIG. 2, the EP catheter interface 112, mapping and guidance system 114, and patch electrode interface 116 are illustrated as separate components. However, in some embodiments, the interfaces 112, 116 and the mapping and guidance system 114 may be components of a single console or computing device with a single housing. In other embodiments, the interfaces 112, 116 and the mapping and guidance system 114 may comprise separate hardware components (e.g., with separate housings) communicatively coupled to one another by electrical cables, wireless communication devices, fiber optics, or any other suitable means of communication. Further, in some embodiments, the EP catheter interface 112 may also function as an interface for the cryoballoon catheter 130 to control inflation of the cryoballoon 132, cooling/heating of the cryoballoon 132, etc. In other embodiments, the cryoballoon catheter 130 is controlled by a separate interface or control system, such as a console.

The mapping and guidance system 114 is coupled to a display device 118, which may be configured to provide visualizations of a cryoablation procedure to a physician. For example, the mapping and guidance system 114 may be configured to generate EP images of the body cavity, visualizations of the propagation of EP waves across the tissue of the body cavity, indications of occlusion by the cryoballoon at an ablation site, or any other suitable visualization. These visualizations may then be output by the mapping and guidance system 114 to the display device 118.

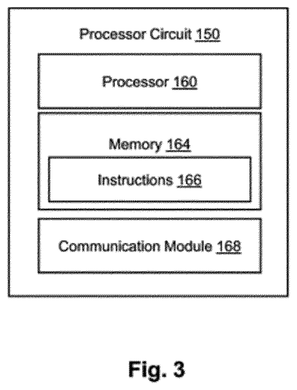

FIG. 3 is a schematic diagram of a processor circuit 150, according to embodiments of the present disclosure. The processor circuit 150 may be implemented in the mapping and guidance system 114, the EP catheter interface 112, the patch electrode interface 116, and/or the display device 118. The processor circuit 150 can carry out one or more steps described herein. As shown, the processor circuit 150 may include a processor 160, a memory 164, and a communication module 168. These elements may be in direct or indirect communication with each other, for example via one or more buses.

The processor 160 may include a central processing unit (CPU), a digital signal processor (DSP), an ASIC, a controller, an FPGA, another hardware device, a firmware device, or any combination thereof configured to perform the operations described herein. The processor 160 may also be implemented as a combination of computing devices, e.g., a combination of a DSP and a microprocessor, a plurality of microprocessors, one or more microprocessors in conjunction with a DSP core, or any other such configuration.

The memory 164 may include a cache memory (e.g., a cache memory of the processor 160), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an embodiment, the memory 164 includes computer-readable medium, which may be a non-transitory computer-readable medium. The computer-readable medium may store instructions. For example, the memory 164, or computer-readable medium may have program code recorded thereon, the program code including instructions for causing the processor circuit 150, or one or more components of the processor circuit 150, to perform the operations described herein. For example, the processor circuit 150 can execute operations described with reference to FIGS. 2 and 4-12, including the operations described with reference to the methods 200, 400. Instructions 166 may also be referred to as code or program code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements. The memory 164, with the code recorded thereon, may be referred to as a computer program product.

The communication module 168 can include any electronic circuitry and/or logic circuitry to facilitate direct or indirect communication of data between the processor circuit 150, the mapping and guidance system 114, the EP catheter 120, the cryoballoon catheter 130, and/or the display 118. In that regard, the communication module 168 can be an input/output (I/O) device. In some instances, the communication module 168 facilitates direct or indirect communication between various elements of the processor circuit 150 and/or the system 100 (FIG. 2). In some embodiments, the processor circuit 150 may further comprise an electrical signal generator and/or an electrical signal measurer configured to control the electrodes of the EP catheter to emit and/or detect electrical signals, including voltages, impedances, and currents.

In one embodiment, the device as defined herein comprises or is a computing device or a computer whether mobile or stationary. Such computing device may take the form of a workstation.

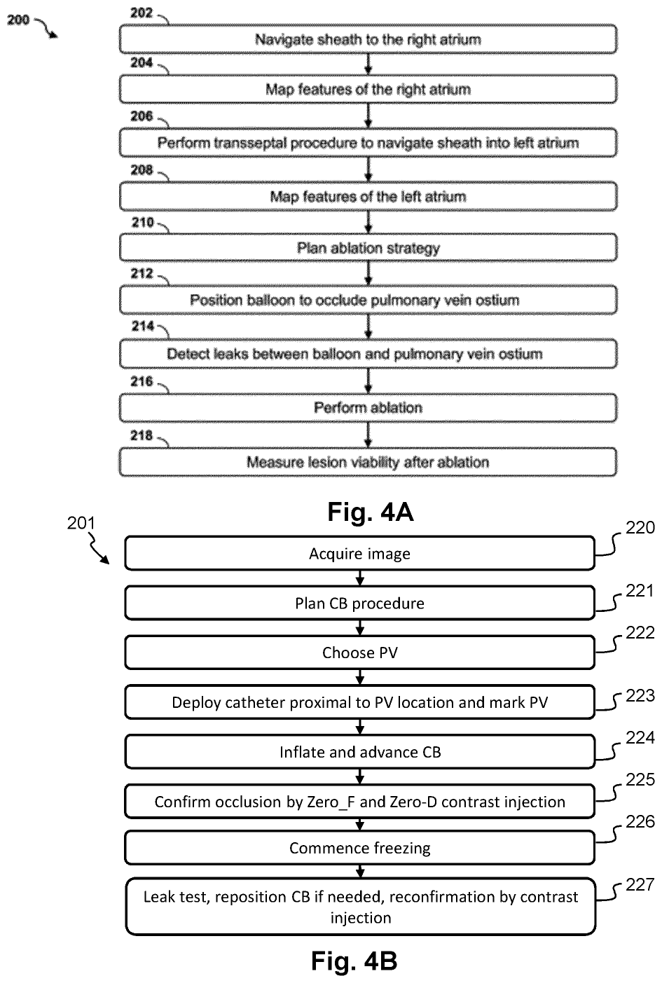

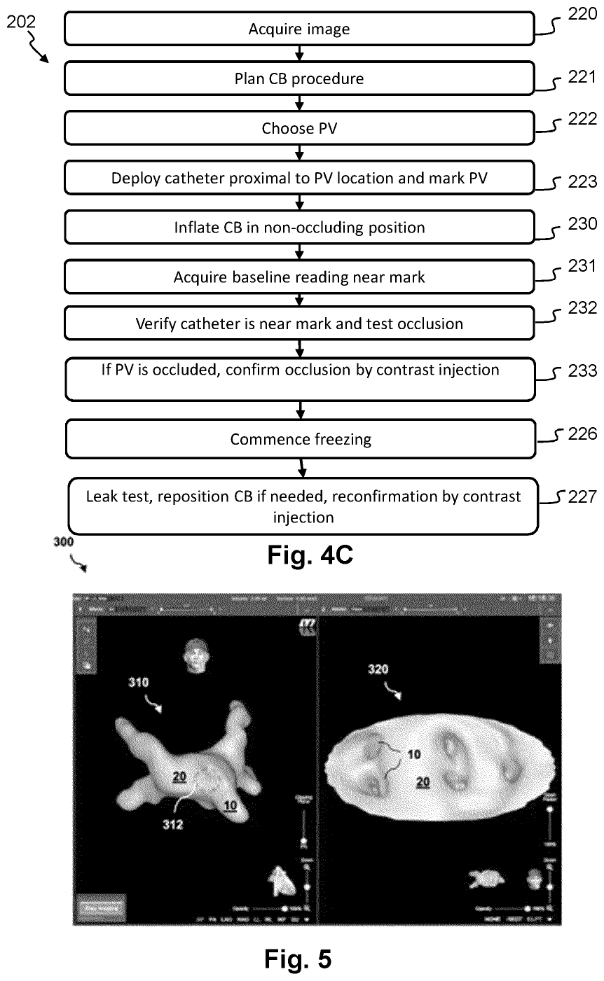

FIG. 4A is a flowchart illustrating a method 200 for performing a balloon ablation procedure, according to some embodiments of the present disclosure. It will be understood that some or all of the steps of the method 200 may be formed using one or more components of the system 100 shown in FIG. 2, including the EP catheter 120, the cryoballoon catheter 130, the mapping and guidance system 114, and/or the patch electrodes 140, 142. In step 202, an EP catheter is navigated to the right atrium. In some embodiments, navigating the EP catheter to the right atrium is performed using EP imaging techniques to image the full path between the access site and the right atrium. For example, in some embodiments, a guidewire with electrodes, a sheath with electrodes, or the EP catheter is inserted into the femoral vein in the patient's leg and navigated to the right atrium while the access route is imaged to guide the placement of the guidewire, sheath, or catheter at the right atrium. In step 204, the right atrium is mapped or imaged by moving the EP catheter within the right atrium. For example, the coronary sinus ostium, septal wall, fossa ovalis, or other features of the right atrium may be imaged using the EP catheter 120.

In step 206, using the images or maps acquired during step 204, the EP catheter is introduced into the left atrium using a transseptal procedure. In some embodiments, the transseptal procedure may involve the use of a transseptal needle or RF needle to penetrate the transseptal wall to introduce the catheter or sheath into the left atrium. In step 208, the features of the left atrium are imaged. For example, the atrial wall, pulmonary vein (PV) ostia, left atrial appendage (LAA), LAA ridge, mitral valve, or other features of the left atrium may be imaged using the EP catheter 120. Anatomical variations can be detected without the use of contrast media. Accordingly, all the necessary anatomical landmarks to perform the ablation procedure can be mapped using the EP catheter and output to a display to guide preplanning of the ablation process and deployment of the balloon. In that regard, FIG. 5 shows a user interface 300 that includes three-dimensional images or views of the left atrium. For example, a first atrial view 310 is a perspective three-dimensional view of the internal structure of the left atrium 20 and pulmonary veins 10. The view 310 includes an EP catheter indicator 312 that shows a location and/or orientation of the electrodes at the distal portion or an EP catheter. In some embodiments, the view 310 can be manipulated (e.g., oriented, re-sized, moved) by a user to provide different views or angles of the left atrium. A second atrial view 320 may comprise a flattened panoramic view of the left atrium 20. For example, the second view 320 may be generated by translating, stretching, distorting, or otherwise modifying the first view 310. The second view 320 shows the ostia of the pulmonary veins in a 3D flattened configuration, which may be advantageous for planning and performing ablation procedures, particularly in order to carry out the full ablation procedure without repeated manipulation and frequent changes in projection of the 3D reconstructed image. The views 310, 320 can be used in step 210 to locate the desired ablation site, and to guide placement of an ablation balloon at the ablation sites, and otherwise plan the ablation procedure.

In step 212, the balloon is positioned at the ablation site (e.g., PV ostium). In some embodiments, positioning the balloon includes inflating or deploying the balloon. In some embodiments, the balloon is positioned at the ablation site using EP imaging to guide the placement. In some embodiments, the processor circuit is configured to output an image of the left atrium and a visualization of the position balloon within the left atrium. For example, in some embodiments, the processor outputs a three-dimensional visual depiction of the balloon and/or the distal portion of the EP catheter that indicates the position and orientation of the balloon within the left atrium. Using these visual depictions, the physician may position and orient the inflated balloon to achieve a full occlusion between the pulmonary vein and the cavity of the left atrium.

An inflated, electrically non-conducting, large cryoballoon naturally represents a substantial spatial interference in the distribution of the electrical fields thereby a baseline recording from the catheter electrodes may be performed with the balloon already inflated but not occluding the pulmonary vein, PV. Advancing the balloon and occlusion of the PV further changes the distribution of the local dielectric properties and when compared to the baseline recording, as long as the catheter remains approximately in the same proximal PV location, optimal balloon apposition in the PV ostia can then be determined.

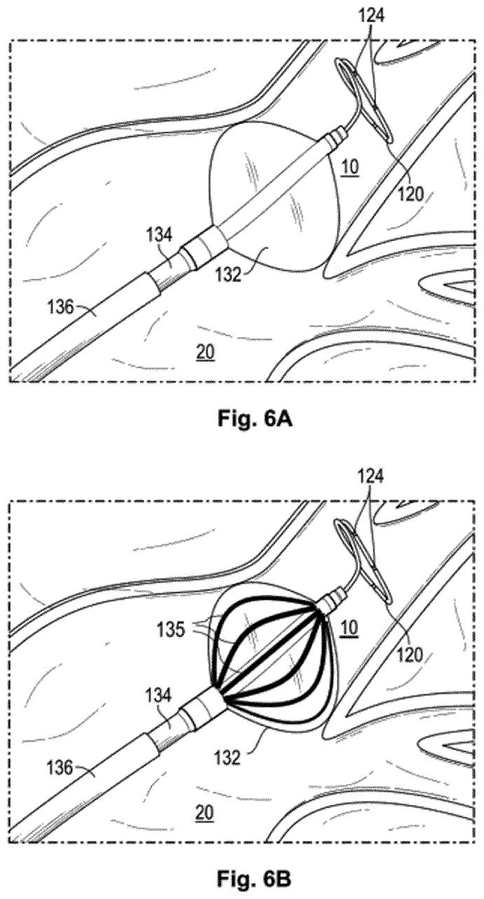

In that regard, FIGS. 6A and 6B are graphical views of inflated ablation balloon 132 positioned at the ostium of the pulmonary vein 10. Referring to FIG. 6A, in an exemplary embodiment, the balloon 132 is cryoballoon. The cryoballoon 132 is configured to be inflated and then at least partially filled with a cooling fluid to cool the cryoballoon to a temperature (e.g., below −65° C.) that causes an electrically-isolating lesion or firewall in the tissue. For example, the cryoballoon 132 may be in fluid communication with a source or reservoir of cooling fluid via one or more fluid lines positioned within a flexible elongate member 134. Further, the cryoballoon 132 may be in fluid communication with an air or gas source, e.g., for balloon inflation, via one or more fluid lines positioned within the flexible elongate member 134. Referring now to FIG. 6B, in other embodiments, the balloon 132 is an RF ablation balloon that includes a plurality of ablation electrodes 135 positioned on an exterior surface of the balloon. The electrodes 135 may be in communication with a processor circuit via one or more electrical conductors, e.g., extending along a length of the flexible elongate member 134. In some embodiments, the processor circuit is proximate to the balloon 132 at the distal portion of the flexible elongate member 134. The processor circuit is configured to selectively activate and control the electrodes to perform an RF ablation procedure. In some aspects, the electrodes 135 may be positioned and spaced around the balloon 132 such that the lesions caused by the electrodes 135 form a contiguous, electrically isolating lesion around an ablation site, such as a pulmonary vein ostium. In some embodiments, the electrodes 135 are distributed around the entire surface of the balloon 132 such that, when the balloon 132 is deployed and positioned to occlude the ablation site, the electrodes 135 are in contact with the tissue around a circumference of the ablation site, such as the pulmonary vein. Further, it will be understood that the illustrated arrangement of electrodes 135 on the RF ablation balloon 132 is merely exemplary, and that the electrodes 135 may be positioned and/or arranged in any suitable configuration to perform a balloon ablation procedure.

Referring to FIGS. 6A and 6B, the balloon 132 may be coupled to a distal portion of a flexible elongate member 134 that is protruding out a distal end of a sheath 136. In some embodiments, the sheath 136 is first introduced into the left atrium, and used to guide the EP catheter 120 to the ablation site. The balloon 132 may then be moved over the EP catheter 120 to the ablation site and positioned such that a full circumference of the balloon 132 is in contact with an entire circumference of the ostium of the pulmonary vein 10, and such that the ablation can be delivered around the entire circumference. In this way, a full occlusion is achieved, which increases the likelihood that the ablation results in complete electrical isolation. By contrast, a partial occlusion in which one or more leaks are present lessens the likelihood of an efficacious treatment. Accordingly, the approaches described in the present disclosure advantageously allow for a guided ablation procedure that can increase the likelihood that the ablation is efficacious.

In some aspects, it may be difficult to ensure, using EP images alone, that full occlusion by the balloon is achieved. For example small leaks may be difficult to detect. Accordingly, in step 214, the EP catheter 120 is used to detect the presence of leaks or gaps in the interface between the balloon 132 and the ostium. As explained above, the EP catheter 120 includes a plurality of internal electrodes 124 at a distal end configured to generate and detect electrical signals used to detect or identify blood flow associated with leakage of blood from the pulmonary vein into the left atrium. Normally, blood flows from the pulmonary vein into the left atrium. Accordingly, a full occlusion of the pulmonary vein should block all blood flow from the pulmonary vein 10 into the left atrium 20. In the method 200, the EP catheter 120 is configured to detect blood flow from the pulmonary vein 10 into the left atrium 20 that should have been blocked by the balloon 132. If an amount of blood flow is detected based on the electrical signals, the system may determine that the placement of the balloon 132 is suboptimal, as it is not fully-occluding. In embodiments, the processor circuit may receive the electrode signals in a data format such that it is capable of processing such data to determine based on the signals indications of an occlusion status of the balloon. Further, in some embodiments, the system may output a visualization indicating such occlusion status which may include for example a location of the gap or leak.

The way the electrical signals from the internal electrodes may be used to detect blood flow is discussed below. The processing of data indicative of such signals to determine occlusion status is also described herein below. The signal processing may be used as such.

However, alternatively or additionally, it is possible to provide further information on occlusion status, such as for example confirmation that there is no leakage, by using an injection of a dielectrically modifying fluid during the occlusions measurements. This can be used to further increase the sensitivity of the PV occlusion test by increasing the signal to noise ratio. Embodiments therefore relate in particular to this monitoring of occlusion status and processing of EP electrode data obtained based on injection of a medium during occlusion.

Advantageously, the medium does not need to be an X-ray contrast agent (Fluoroscopic dye), but may be a less harmful and less viscous saline or dextrose-in-water (D5W) fluid. This shortens the required test time and enables a zero-fluoroscopy (zero-F) and zero-dye (zero-D) procedure. The medium used in embodiments will herein after be referred to as dielectric medium in the sense that it is a medium that has dielectric properties different from that of normal blood. An example of such property may be electrical conductivity which for normal blood is ˜6.6 mS/cm. The dielectric medium may thus have an electrical conductivity different form that of normal blood.

Referring to FIG. 4A, in step 216, if it is determined that there are no leaks or gaps such that the balloon is adequately occluding blood flow from the pulmonary vein, the ablation procedure may be performed. However, in some instances, a physician may decide to proceed even though a leak is detected. In some embodiments, the ablation procedure is a cryoablation procedure that involves cooling an inflated cryoballoon, using a fluid, to a temperature sufficiently cold to create a lesion at the ablation site. In some embodiments, the cooled cryoballoon is left in place for 2-5 minutes to complete the lesion. However, in other embodiments, the cryoballoon may be left in place for any suitable amount of time, both longer and shorter. In some embodiments, the ablation procedure is an RF balloon ablation procedure.

Even though the balloon may be determined to be fully occluding before or during the ablation procedure, in some instances, the resulting lesion does not fully isolate the pulmonary vein from the left atrium. For example, in some instances, the balloon may move during the ablation procedure. In some instances, even though the balloon fully occludes the pulmonary vein, other aspects may cause the lesion to have gaps around the ablation site such that the lesion does not fully isolate the pulmonary vein, including insufficient ablation time, insufficient contact or pressure of the balloon on the tissue, etc. Accordingly, in some aspects, it may be beneficial to perform a post-ablation verification procedure to test or measure the effectiveness of the ablation procedure. In that regard, in step 218, the system determines or measures lesion viability using the EP catheter. If a cryoballoon is used, the cryoballoon is reheated to detach the cryoballoon from the ablated tissue to determine lesion viability. The determination of lesion viability may include a lesion visualization procedure in which the electrodes 124 of the EP catheter 120 are used to compute transmurality and/or permanency of the lesion, which may be assessed immediately after ablation.

Accordingly, the procedures described in the method 200 allow for a complete balloon ablation procedure to be performed without the use of fluoroscopy, contrast agent, or ultrasound to guide the procedure, and allow for reliable post-ablation validation to reduce the need for re-do procedures to address incomplete lesions.

FIG. 4B is a flowchart for a method 201 showing how injection of a dielectric modifying fluid is used to improve the signal to noise ratio for leak detection.

In step 220, the imaging is carried out, for example generating 3D and panoramic images.

In step 221, the images are analyzed and the cryoballoon procedure is planned.

In step 222, the pulmonary vein is chosen for isolation.

In step 223, the catheter is deployed proximal to the PV position. The PV location is marked on the image, creating a shadow.

In step 224, the balloon is inflated and is then advanced to the occluding position.

In step 225, the occlusion is confirmed by injecting a dielectric modifying fluid such as saline solution or dextrose solution.

In step 226, freezing is commenced if the PV is fully occluded.

In step 227, if there is a small residual leak, the balloon is repositioned and occlusion is reconfirmed with the assistance of a dielectric modifying injection.

FIG. 4C is a flowchart for another method 202 using injection of a dielectric modifying fluid to improve the signal to noise ratio for leak detection.

Steps 220 to 223 are the same as in FIG. 4B.

In step 220, the imaging is carried out, for example generating 3D and panoramic images.

In step 221, the images are analyzed and the cryoballoon procedure is planned.

In step 222, the pulmonary vein is chosen for isolation.

In step 223, the catheter is deployed proximal to the PV position. The location is marked on the image, creating a shadow.

In step 230, the balloon is inflated in a non-occluding position.

In step 231, a baseline reading with the inflated balloon near the shadow mark.

In step 232, it is verified that the catheter is near the shadow mark and the occlusion is tested. This occlusion testing relies on the baseline acquisition and is without use of a contrast medium. It for example uses detection of a change in the native catheter signals and other features (such as a pulse pattern and respiration signal, and the catheter location within the imaging coordinate system).

In step 233, the occlusion is confirmed by injecting a dielectric modifying fluid such as saline solution or dextrose solution.

Steps 226 and 227 are the same as in FIG. 4B.

In step 226, freezing is commenced if the PV is fully occluded.

In step 227, if there is a small residual leak, the balloon is repositioned and occlusion is reconfirmed with the assistance of a dielectric modifying injection.

Another possible method is to start with a baseline acquisition and gradually approach the occlusion position. The contrast injection is then used to confirm the position.

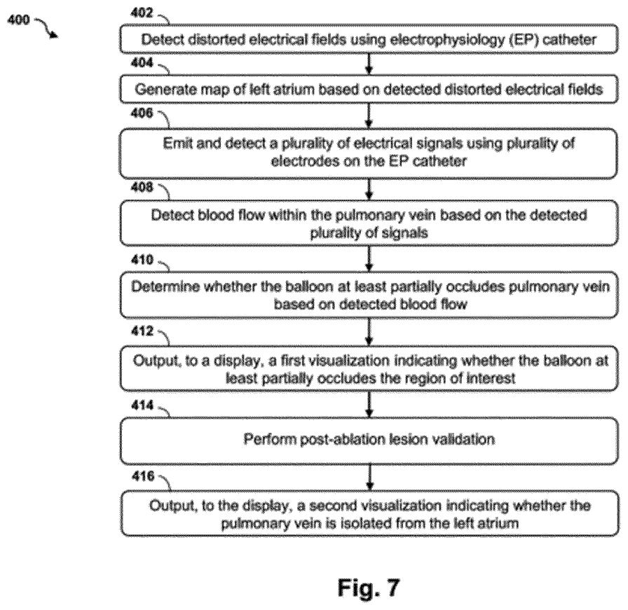

FIG. 7 is a flowchart showing a method 400 for assessing an occlusion at an ablation site by an ablation balloon using an EP catheter. As described above, the ablation balloon may comprise a cryoballoon, an RF ablation balloon, or any other suitable type of ablation balloon. In some aspects, some or all of the steps of the method 400 may be used to carry out the methods 200 shown in FIGS. 4A and 4B. For example, the method 400 may be used to perform the step 214 in the method 200 and the step 227 in the method 201. In step 402, the electrodes of the EP catheter detect distortions in electrical fields generated by external patch electrodes placed on the body of a patient. The distortions in the electrical fields may be caused by the shape and structure of the tissue, and thus the detected distortions can be used to generate a map of the left atrium in step 404. In some embodiments, the map may comprise a three-dimensional volumetric image of a body cavity, such as the left atrium. In some embodiments, the map may comprise a flattened panoramic view, as shown in FIG. 5, for example. In step 406, with the balloon positioned and deployed at the ablation site, the electrodes of the EP catheter are activated to generate a plurality of signals and detect the plurality of signals. In that regard, in some embodiments, each electrode is configured to emit an electrical signal at a particular frequency. Further, all of the electrodes may be configured to detect the electrical signals emitted by the other electrodes at the other frequencies, in addition to the electrical signal emitted by the electrode. For example, a first electrode may be configured to emit a first electrical signal at a first frequency, and a second electrode may be configured to emit a second electrical signal at a second frequency. In an exemplary embodiment, the first electrode is used to detect the first electrical signal at the first frequency, and the second electrical signal is used to detect the second electrical signal at the second frequency. However, in other embodiments, the first electrode may detect the second electrical signal at the second frequency in addition to its own first electrical signal at the first frequency. Similarly, the second electrode may detect the first electrical signal at the first frequency in addition to its own second electrical signal at the second frequency.

In step 408, based on the detected electrical signals, the system detects blood flow (or the lack of blood flow) in the pulmonary vein. In that regard, referring to FIGS. 6A and 6B, the electrodes 124 at the distal end of the EP catheter 120 are positioned within blood pool in the pulmonary vein 10. When the pulmonary vein is fully occluded by the balloon, there is no blood flow into the left atrium. Blood flow affects the electrical signals detected by the electrodes 124.

For example, in some aspects, blood flow may add noise to one or more electrical signals, change the average amplitude of the electrical signals, or otherwise affect the electrical signals in a way that can be detected by the system.

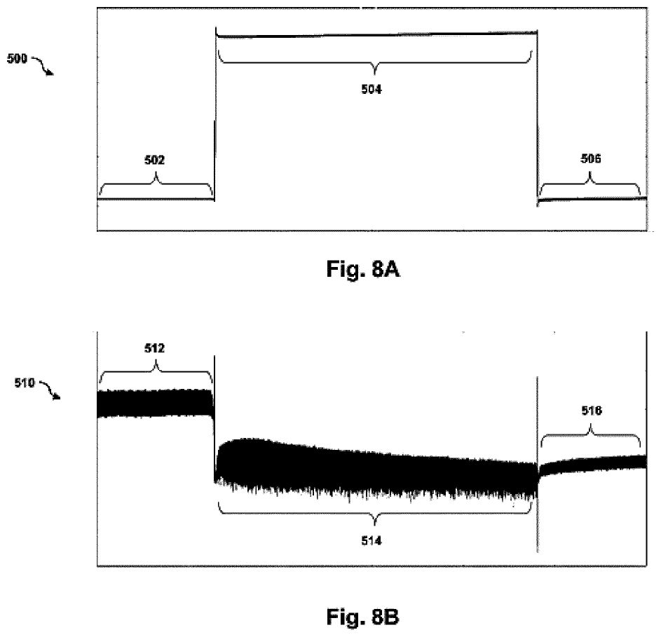

This detection may be performed without any injection of a dielectric medium. Additionally or alternatively, the detection makes use of the response of the the electrical signals to the injection of the dielectric medium. Before such embodiments are described, the way occlusion can be detected without the need for a dielectric medium injection to improve signal to noise ratio will be explained with reference to FIGS. 8A and 8B.

FIGS. 8A and 8B are graphical views of electrical signals detected or measured by an electrode distance from a leak, and an electrode near a leak, respectively. In that regard, FIG. 8A shows a graph 500 of potentials for a first electrode which is positioned distant from a leak. In a first portion 502 of the graph 500, the balloon is not occluding the pulmonary vein. In a second portion 504 of the graph 500, the balloon occludes the pulmonary vein but leaves a small leak distant from the first electrode. In a third portion 506 of the graph 500, the balloon is moved such that it does not occlude the pulmonary vein. It is observed that the first graph 500 of potentials in FIG. 8A shows significant changes in average amplitude when transitioning between the first 502, second 504, and third 506 portions of the graph 500. In some aspects, the significant changes in the potentials indicate that there is adequate regional occlusion at least in the area near the first electrode, which manifests by a marked change in voltage in the second portion 504 relative to the first 502 and third 506 portions.

By contrast, FIG. 8B shows a graph 510 of potentials for a second electrode that is positioned adjacent a leak. Similar to the graph shown in FIG. 8A, the graph in FIG. 8B includes a first portion 512 in which the balloon is not occluding the pulmonary vein, a second portion 514 in which the balloon occludes the pulmonary vein but leaves a leak near the second electrode, and a third portion 516 where the balloon does not occlude the pulmonary vein. It is observed that the change in the potentials of the second electrode between the first 512, second 514, and third 516 portions of the graph 510 is less pronounced, which indicates that there was blood flow during the second portion 514 (the occlusion portion), just as there was blood flow during the first 512 and third 516 portions in which the balloon did not occlude the pulmonary vein. According to these observations, the system may analyze the electrical signals to determine whether a leak is present near a given electrode. For example, a processor circuit, such as the processor circuit 150 shown in FIG. 3, may perform the analyses, including those described above in steps 406 and 408. For example, the processor circuit may include an electrical signal generator and/or an electrical signal measurer configured to control the electrodes of the catheter to emit and/or detect electrical signals. In some aspects, the processor circuit may be configured to determine whether there is a leak based on an amount of change in the signal between a first period of no occlusion, and a second period of supposed occlusion. For example, in some embodiments, step 408 may include determining a pre-occlusion baseline signal measurement for each electrode when the balloon is not occluding the pulmonary vein. Subsequently, when it is determined that at least one electrode is in an area of occluded blood flow, the processor circuit can determine an amount of change, for each electrode, between the baseline measurement and the current measurement. Little or no change between the baseline and the current measurement may indicate that blood flow is present near the electrode. In some aspects, the processor circuit may be configured to compare the change in the electrical signals before and after occlusion to a threshold and/or the other electrical signals to identify dissimilar behaviors in the electrical signals. For example, the processor circuit may analyze changes in amplitude, noise, variation in amplitude, and other features of the electrical signals to determine that at least regional occlusion in an area near a given electrode if the change between the baseline signal and the signal obtained during occlusion rises above a threshold. In some embodiments, identifying leaks may include using logarithmic analysis of the electrical signals and computing the area under the curve for each electrode to highlight signals likely associated with a leak.

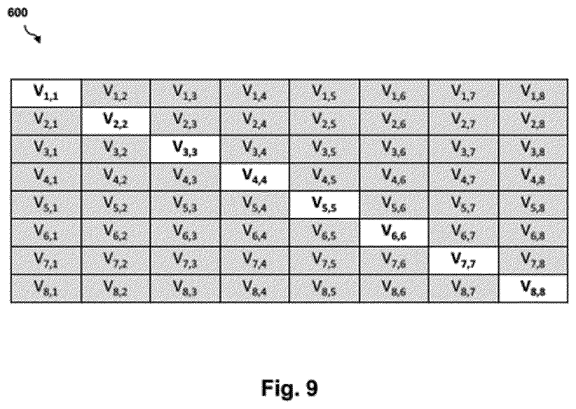

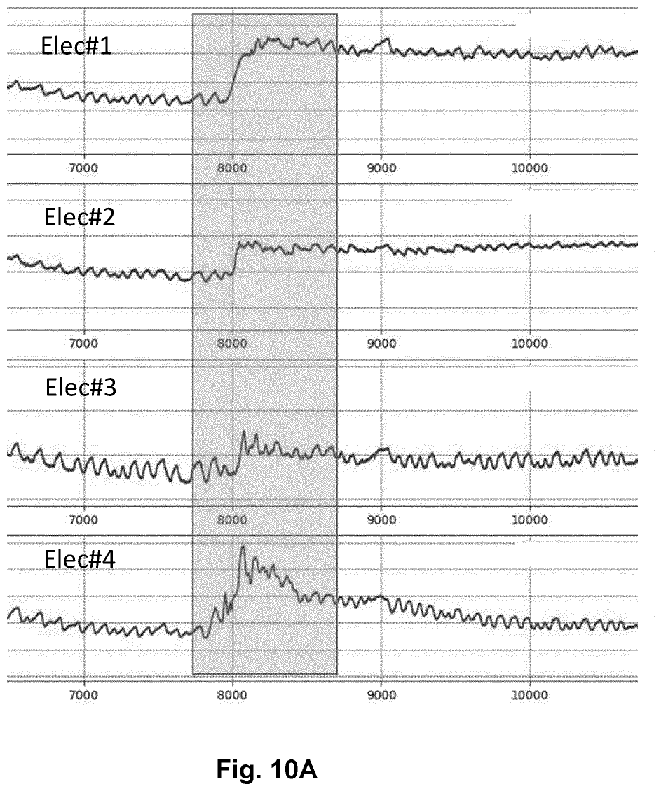

FIG. 9 is a graphical view of a Natives matrix 600 representing the signals analyzed by the processor circuit to detect blood flow as described with respect to steps 406 and 408, according to one embodiment of the present disclosure. The voltages of each block are defined as V_{i, j}, where i represents which electrode is receiving, and j represents the transmitting electrode, or the frequency at which the ith electrode is measuring the signal. Thus, V_{3, 5}, for example, is the voltage measured by electrode number 3 at the frequency of electrode number 5. In the table of FIG. 9, the voltages, or electrical signals, that are used to detect blood flow are not shaded, and the voltages that are not used are shaded. Thus, the voltages that are used comprise a diagonal of the matrix 600, which are the electrical signals that are generated and detected using the same electrodes. For example, the signal V_{1, 1}, which is the signal generated and received by the first electrode, is used to detect blood flow, while V_{1, 2}, may not be used. However, in other embodiments, any suitable combination of voltages is used. For example, some or all of the non-native voltages are used to detect blood flow. For example, in some embodiments, a mirror-image diagonal of voltages is used in addition to the diagonal in the matrix 600 of FIG. 9. Further, in some embodiments, current and/or electrical impedance are used instead of, or in addition to voltage. For example, in some embodiments, the system may be configured to compare impedance measurements to detect flow at a given electrode. Further, although the matrix 600 is an 8-by-8 matrix corresponding to an eight-electrode EP catheter, the matrix 600 of electrical signals may include any suitable number of signals, both larger or smaller. For example, as described above, in some embodiments, 2, 4, 6, 8, 10, 12, 15, 20, 30, 60, or any other suitable number of electrodes may be used to detect blood flow. Accordingly, in some embodiments, the matrix 600 may include a 2-by-2 matrix, a 4-by-4 matrix, a 10-by-10 matrix, a 20-by-20 matrix, a 60-by-60 matrix, or any other suitable matrix.

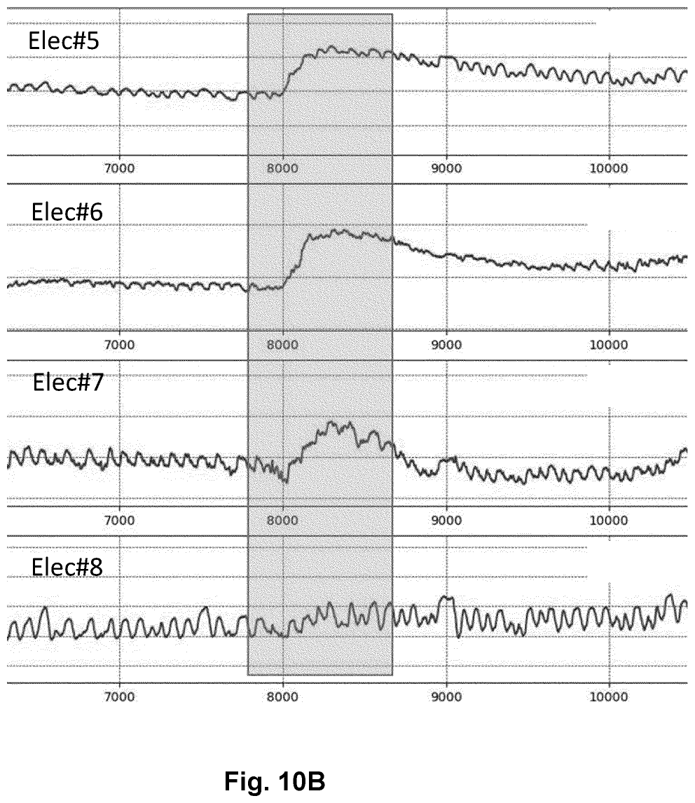

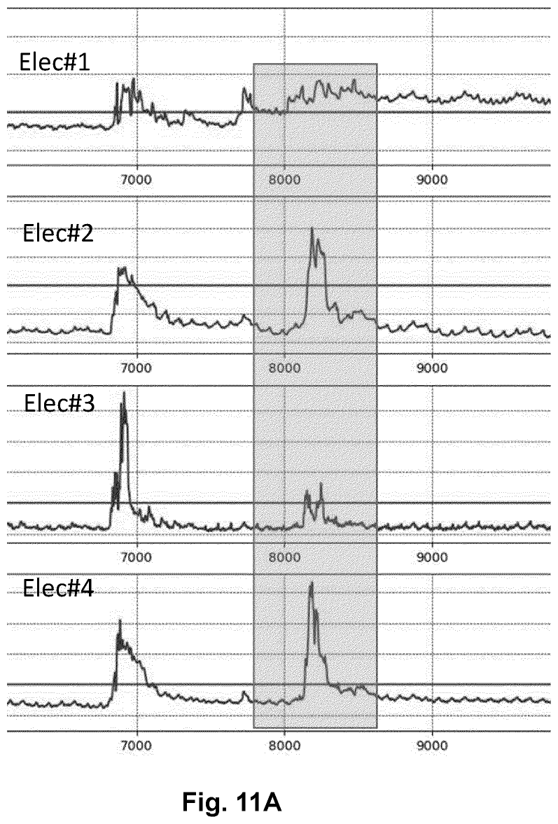

FIGS. 10 to 13 show the internal electrode signals from an 8 electrode catheter at different levels of occlusion, obtained during an experimental test using a known Fluoroscopic dye Omnipaque™ marketed by General Electric™ as the dielectric medium. For each experiment 6-10 ml due was injected using an automated injector with appropriate rate and pressure settings. While manual injection is possible, automated injection was found to be beneficial for reproducibility of experiments. In each case the first (“A”) figure shows the signals for electrodes 1 to 4 (from top to bottom) and the second (“B”) figures shows the signals for electrodes 5 to 8 (from top to bottom). The highlighted area is the measurement time period. There is a preceding pre-injection time, used for comparison purposes. It for example has a 5 second duration. The subsequent measurement time period is immediately after the injection, and for example has a 10 second duration.

It has been found that for situations where there is a leak, the electrode signal at which the leak is detected has a high amplitude peak with a rapid decay back to the pre-injection level. When there is occlusion, there is a less marked peak, and also slower decay. Furthermore, the value remains above the pre-injection level at the end of the measurement window.

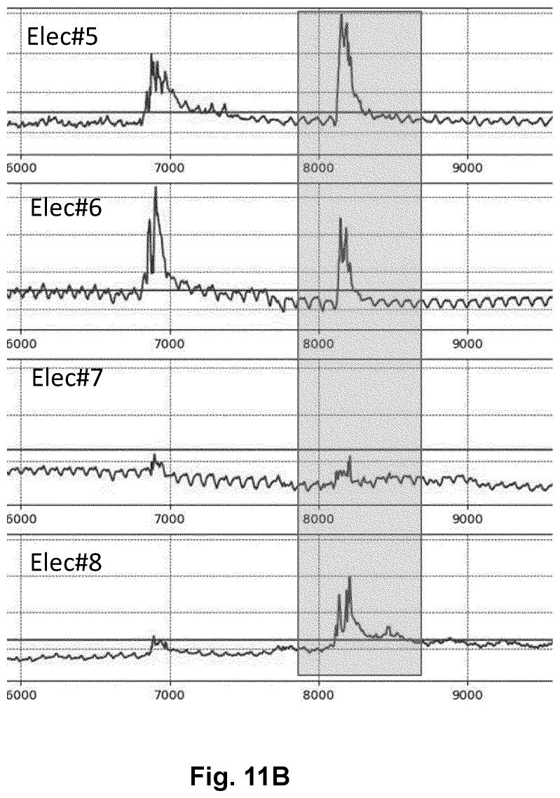

FIGS. 10A and 10B show internal electrode signals when there is full occlusion. For each of electrodes 1 to 6, occlusion is seen as a relatively small signal rise (compared to FIGS. 11 to 13), and which does not return rapidly to the previous baseline level, or returns slowly. The injected medium for example does not reach electrode 8, and electrode 7 shows a less characteristic response.

FIGS. 11A and 11B show internal electrode signals when there is a large residual leak. The more pronounced peak can be seen, and the level returns to the pre-injection level. Electrodes 2 to 7 all show the characteristic signal properties associated with a leak. Only electrode 8 has a single which does not return to the pre-injection level and thus indicates occlusion.

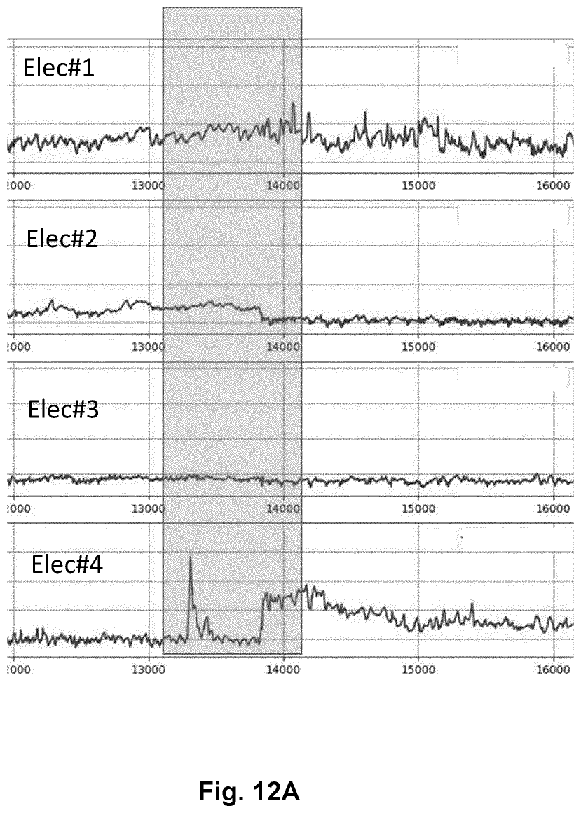

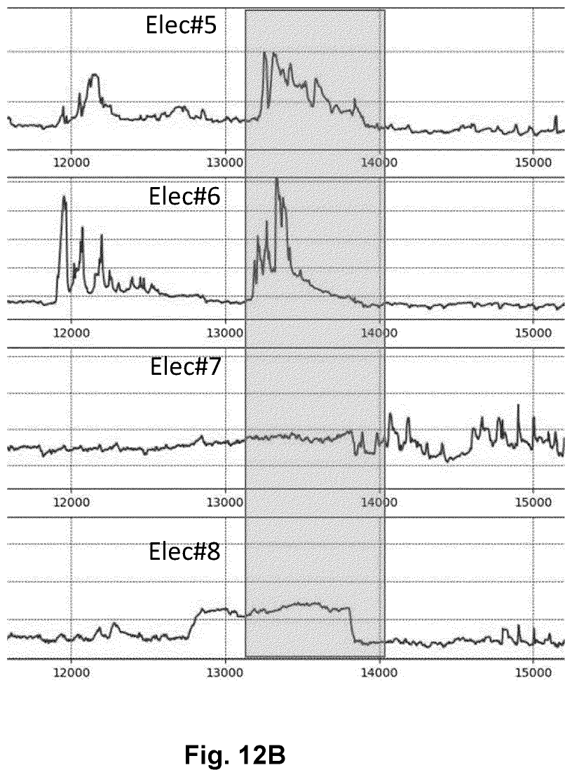

FIGS. 12A and 12B show internal electrode signals when there is a moderate residual leak. The injected medium reaches electrodes 4, 5 and 6, which all show a moderate residual leak.

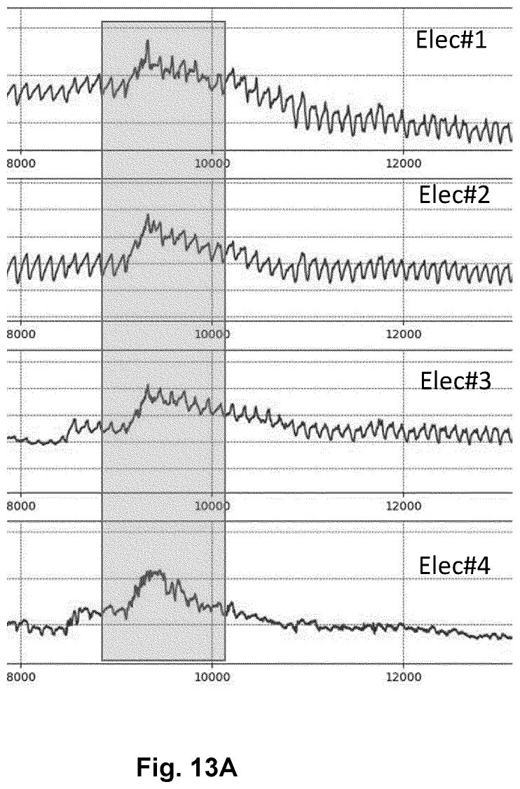

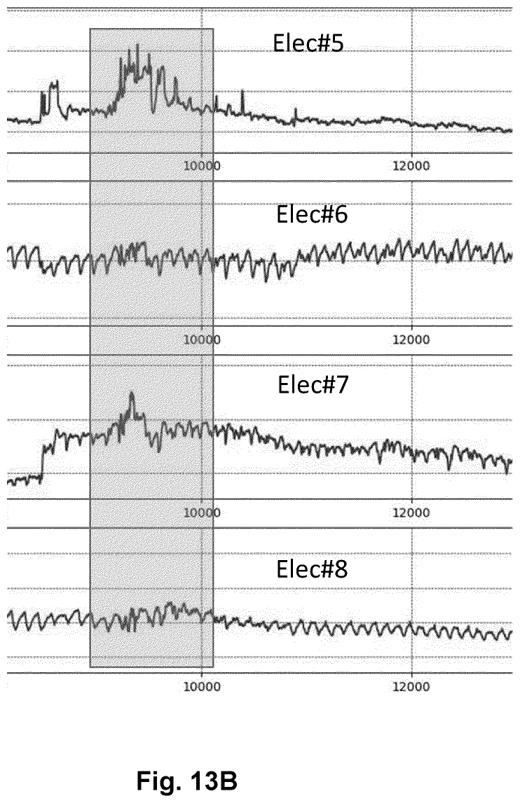

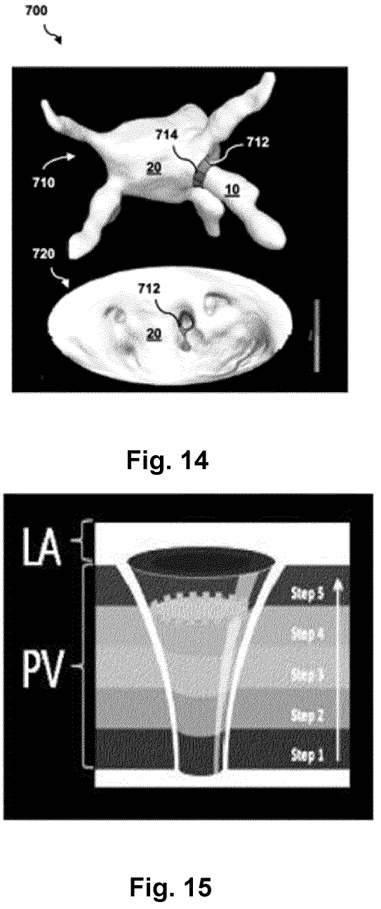

FIGS. 13A and 13B show internal electrode signals when there is a small residual leak. The medium reaches electrodes 1 to 7. Electrodes 1 to 4 indicated occlusion, and electrodes 5 to 7 indicate a small residual leak.

By analyzing the signals from multiple of all of the full set of electrodes, e.g. following a spiral or circular (lasso) shape, a leakage level can be graded and can also be spatially located by comprehensively assessing all electrodes in 2D or even 3D. The detection may thus resolve between multiple small leaks at different angular positions or a single larger leak, for example.

Thus, the leak detection in particular or occlusion status in general is based on one or more electrical signals measured with one or more electrodes of an EP catheter located within a pulmonary vein during an injection of a dielectric medium in the pulmonary vein distal to the balloon during an occlusion event of for example an ablation procedure. The signal analysis is based on the response of the one or more detected electrical signals to the injection of the medium. In this way the response, e.g. the temporal response (i.e. the way that medium flows through the circulatory system) to the injection of the medium is monitored. Thus once the systems are obtained by the system and converted to appropriate data format, the processing circuit may perform the necessary processing to derive the occlusion status form the data indicative of the response.

In embodiments, the response of one or more of the detected plurality of electrical signals in response to injection of said medium comprises one or more of:

the amplitude of a signal spike;

the time decay of a signal spike; and

a comparison of the signal amplitude before and after a signal spike.

The signal amplitude before the signal spike is for example based on a measurement window before the injection and the amplitude after the signal spike is at the end of a measurement window, where the end of the measurement window may be chosen by the user, or may be preset or may be determined automatically. For example, the end of the window may correspond to decay of the spike to half maximum or some other chosen amplitude value. The end of the window may also correspond to a time stamp where the amplitude of the signal has become substantially stable over time or even have returned to a level substantially equal to the level before injection. The end of the window can correspond to e.g. of maximum duration.

The electrical signals are all gathered distally of the ablation balloon in the proximal PV position, i.e. in the cavity that is to be or is occluded by the balloon during the procedure. There are no comparison electrodes at the proximal side of the balloon, so the electrode signal analysis is based solely on the dynamic changes in dielectric properties distally beyond the balloon, i.e. within the PV 10, PV 10 being an example of such cavity.

The occlusion analysis may also provide additional clinically relevant warnings, such as based on the following measurements and warnings:

(i) Distance To Ostium (DTO)—the catheter is too distal in the vein;

(ii) Distance To Shadow (DTS)—the catheter is too far from the shadow;

(iii) Distance To Mesh (DTM)—the catheter is outside the imaged mesh.

The conductivity of the injected dielectric medium may be higher or lower compared to the blood conductivity of around 6.6 mS/cm. Preferably the conductivity of the dielectric medium is lower than that of blood. It has been observed that signal to noise ratios of the measurements are then increased with respect to measurements that do not use a contrast agent. The dielectric medium for example has a conductivity of below 1 mS/cm, for example below 0.1 mS/cm and preferably below 0.05 mS/cm. Whether higher or lower, the larger the difference between the conductivity of blood and the medium, the higher the effect (for example the higher the amplitude spike) and the higher the improvement of signal to noise ratio may be. The dye, such as for example the Omnipaque™) has a conductivity of blood and the medium, the higher the The dye, such as for example the Omnipaque™) has a conductivity lower than blood of ˜0.01 mS/cm. The data of FIGS. 10 to 13 have been obtained using a Fluoroscopic agent marketed under the tradename Omnipaque™ which contains Iohexol in water as the active X-ray absorbing compound. Iohexol is N,N′-Bis(2,3-dihydroxypropyl)-5-[N-(2,3-dihydroxypropyl)-acetamido]-2,4,6-triiodoisophthalamide, which is a nonionic, water-soluble radiographic contrast medium with a molecular weight of 821.14 (iodine content 46.36%). In aqueous solution each triiodinated molecule remains undissociated and is a non-ionic compound which may account for the low conductivity of the Omnipaque™. Another non-ionic medium may comprise glucose also known as dextrose which also is a non-ionic compound. A 5 weight % solution in water (D5W) has a conductivity of around 0.01 mS/cm. The above does not mean that agents with higher conductivity than blood cannot be used with the method and system as described herein. For example, 0.9% saline is known to be a medium that has a higher conductivity (˜14.5 mS/cm) than blood and it may be used with the methods and systems disclosed herein. Saline is also known to provide a contrast agent in impedance-based occlusion assessment as disclosed in U.S. Pat. Nos. 10,595,922 or 9,956,025. However, the increased signal-to-noise ratio referred to hereinbefore was not observed with the use of saline with the methods and systems described herein. Without wanting to be bound by theory this is likely because the conductivity difference with blood is not high enough. It is in general likely difficult to obtain a dielectric medium with a conductivity that is more than a factor of 10 higher than that of blood for the injection purpose.

Referring again to FIG. 7, in step 410, based on the detected blood flow (or lack of blood flow), the system determines whether the balloon at least partially occludes the pulmonary vein. In that regard, in some embodiments, the system is configured to determine whether the balloon at least partially occludes the pulmonary vein by determining whether any gaps are present in the interface between the balloon and the ablation site, and/or the extent of the gaps. For example, the system may determine that full occlusion is achieved if none of the electrical signals from the electrodes indicate blood flow. By contrast, the system may determine that the occlusion is not complete, and that one or more leaks are present, if blood flow is detected at any of the electrodes. Further, in some embodiments, the system may determine the location of any leak(s) in the interface by determining which detected electrical signals indicate blood flow.

In step 412, the system is configured to output, to a display, a first visualization indicating whether the balloon occludes the region of interest. In some embodiments, the first visualization includes an indicia of full occlusion or no occlusion. For example, text, check marks, colored boxes, or other visual indicators can be used to inform the physician whether full occlusion has occurred. In some embodiments, the first visualization indicates which parts of the interface between the balloon and the ablation site are occluded, and which parts are not. For example, in some embodiments, the first visualization may be overlaid on a map of the body cavity to identify the locations of any leaks on corresponding locations in the map. In some embodiments, the first visualization may appear similar to the indicator 312 shown in FIG. 5, with highlighting applied to the number of an electrode that is near a leak. In some embodiments, the first visualization is configured to indicate an extent or degree of a leak and/or occlusion, such as an angular portion of the pulmonary vein-balloon interface that includes leaks. In some embodiments, the first visualization may indicate the amount of flow or extent of the leak at a given location. By indicating the extent or degree of the leak, the physician may use their knowledge and discretion to decide whether to reposition the balloon before proceeding, or simply begin the ablation procedure and make adjustments to complete the ablation.

In that regard, FIG. 11 is a graphical view of a user interface 700 used to indicate leaks in an interface between an ablation balloon and an ostium of a pulmonary vein 10. The user interface includes a three-dimensional volumetric view 710 of the left atrium 20, with a first visualization 712 overlaid on the ostium of the pulmonary vein 10. The first visualization includes a plurality of indicators corresponding to each of a plurality of electrodes used to detect leaks in the interface. A color or shade of the indicators may be representative of the presence, absence, or degree of leakage. For example, the first visualization 712 includes an indicator 714 that identifies a location of a leak. Thus, a first shade of the indicators may represent that no leak is detected in the corresponding area, and a second shade (i.e., the shade of the indicator 714) indicates a leak. In some embodiments, the first visualization 712 may be a continuous ring overlaid on the map to identify varying levels of leakage between the pulmonary vein and the left atrium. For example, in some embodiments, the measurements of the electrodes are interpolated spatially to generate the continuous ring indicator. In other embodiments, the first visualization 712 may include a marker overlaid over an area in which a leak is detected, if any. A flattened panoramic view 720 of an interior of the left atrium 20 is also shown below the volumetric view 710. The flattened panoramic view 720 may comprise a modified arrangement of the data used to generate the volumetric view 710. The panoramic view 720 also includes the first visualization 712 overlaid on the ostium of the pulmonary vein to similarly identify locations of any leaks in the pulmonary vein-balloon interface.

Referring again to FIG. 7, once the balloon is placed to fully occlude the ablation site, indicated by the first visualization, the ablation can be performed. After performing the ablation, it may be desirable assess the completeness of the isolation caused by the ablation. Accordingly, in step 414, a post-ablation lesion validation procedure is performed. In conventional approaches, an electrical isolation test is used that involves pacing an electrode distal of the lesion site, and another electrode proximal of the lesion site, to identify areas in the lesion that are not electrically-isolating. However, those procedures may suffer from reduced reliability. In that regard, ablation procedures are known to cause edema around the lesion site during and immediately after the ablation procedure. The edema is a buildup of fluid caused in response to the creation of the lesion. The edema is electrically isolating, but dissipates after the creation of the lesion. Thus, conventional isolation tests may indicate a lesion as fully isolating, even though the lesion eventually returns to a pre-edema state that leaves gaps in the lesion. Accordingly, the present disclosure provides an approach for validating a lesion using a lesion visualization procedure. One aspect of the lesion visualization procedure is shown in FIG. 12. In that regard, with the EP catheter placed within the ostium of the pulmonary vein, the EP catheter is retracted in a step-wise fashion from step 1, which is the most distal position within the pulmonary vein, to step 5, which is the most proximal position closest to the left atrium. However, fewer or more steps may be used during the lesion visualization procedure, including 2, 4, 6, 8, 10, 20, or any suitable number, both larger and smaller. At each steps 1-5, the EP catheter acquires lesion validation data that can assess one or more aspects of the lesion and/or electrical isolation between the pulmonary vein and the left atrium. In some aspects, performing the lesion validation includes a lesion visualization procedure that measures transmurality and/or permanency of the lesion. Additional details of measuring transmurality and/or permanency can be found in, for example, U.S. Publication No. 2018/0125575, titled “Lesion Assessment by Dielectric Property Analysis,” the entirety of which is hereby incorporated by reference.

Based on the lesion validation data, the processor may determine whether the lesion likely electrically isolates the pulmonary vein from the left atrium, of if gaps exist in the lesion that allow for AF to return. In step 416, a second visualization is output the display to indicate whether the lesion is isolating. As shown in the user interface 800 of FIG. 13, in some embodiments, the second visualization 812 comprises a marker overlaid on a three-dimensional volumetric view 810 of the left atrium 20 and the pulmonary vein 10. The second visualization 812 may be similar in some aspects to the first visualization 712 shown in FIG. 11. For example, the second indicator may comprise an annular image object overlaid on the surface of the view 810 that indicates whether a given area of the ablation site is electrically isolating. As shown in FIG. 13, when a gap in the lesion is detected, the second visualization 812 includes an indicator 814 that identifies the location of the gap. In some embodiments, the second visualization includes a number of regions corresponding to a number of electrodes on the catheter. In other embodiments, the second visualization 812 may be divided into angular portions irrespective of the number of electrodes. Further, in some embodiments, the second visualization 812 may include a continuous indication of electrical isolation at the ablation site, which may be generated by interpolating the measurements obtained by each electrode of the EP catheter. The user interface 800 further includes a flattened panoramic view 820 of the interior of the left atrium 20. Similar to the view 810, the flattened panoramic view 820 includes the second visualization 812 comprising an indicator 814 showing the location of a gap in the lesion.

It will be understood that one or more of the steps of the methods 200 and 400, such as generating the map of a body cavity, detecting blood flow within a body cavity or lumen using electrical signals acquired by an EP catheter, and outputting visualizations to a display indicating whether the balloon fully occludes an ablation site, can be performed by one or more components of an EP-guided ablation system, such as a processor circuit of a mapping and guidance system, an EP catheter, a cryoballoon catheter, an RF ablation balloon catheter, external body patch electrodes, or any other suitable component of the system. For example, the described ablation procedures may be carried out by the system 100 described with respect to FIG. 2, which may include the processor circuit 150 described with respect to FIG. 3. In some embodiments, the processor circuit can use hardware, software or a combination of the two to perform the analyses and operations described above. For example, the result of the signal processing steps of the methods 200 and 400 may be processed by the processor circuit executing a software to make determinations about the locations of the electrode in the 3D image, etc.

It will also be understood that the embodiments described above are exemplary and are not intended to limit the scope of the disclosure to a given clinical application. For example, as mentioned above, the devices, systems, and techniques described above can be used in a variety of balloon ablation applications that involve occlusion of a body cavity or body lumen. For example, in some embodiments, the techniques described above can be used to guide a cryoablation procedure using a cryocatheter comprising a cryoballoon as described above. In other aspects, the techniques described above can be used to guide an RF ablation procedure in which a plurality of RF ablation electrodes positioned on the surface of an inflatable balloon are used to create an electrically-isolating lesion in cardiac tissue. For example, the HELIOSTAR RF balloon catheter, manufactured by Biosense Webster, Inc., includes 10 ablation electrodes positioned on an external surface of the inflatable balloon, and 10 electrodes on a circular mapping catheter positioned distally of the balloon and configured to be positioned inside the pulmonary vein. Alternatively, or additionally, the electrodes positioned on the RF ablation balloon can be used to determine the contact force or tissue pressure of the balloon on the tissue to detect online potential inadvertent balloon dislodgement during the ablation process. Techniques for using electrodes to determine contact force against tissue are described in U.S. Patent Application Publication No. 2018/0116751, titled “Contact Quality Assessment by Dielectric Property Analysis,” the entirety of which is hereby incorporated by reference. Further, while the ablation procedures are described with respect to the heart and associated anatomy, it will be understood that the same methods and systems can be used to guide ablation procedures in other body volumes, including other regions of interest in the heart, or other body cavities and/or lumens. For example, in some embodiments, the EP guided ablation procedures described herein can be used to guide treatment procedures in any number of anatomical locations and tissue types, including without limitation, organs including the liver, heart, kidneys, gall bladder, pancreas, lungs; ducts; intestines; nervous system structures including the brain, dural sac, spinal cord and peripheral nerves; the urinary tract; as well as valves within the blood, chambers or other parts of the heart, and/or other systems of the body. The anatomy may be a blood vessel, as an artery or a vein of a patient's vascular system, including cardiac vasculature, peripheral vasculature, neural vasculature, renal vasculature, and/or any other suitable lumen inside the body. In addition to natural structures, the approaches described herein may be used to examine man-made structures such as, but without limitation, heart valves, stents, shunts, filters and other devices. in the kidneys, lungs, or any other suitable body volume. Further, the occlusion and flow detection features described above can be employed in various applications to determine flow occlusion. For example, the flow occlusion detection procedures described above can be used in the diagnosis and/or treatment of aneurisms, stent deployment, and any other suitable application.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

1. A device for assisting a balloon ablation therapy procedure using an ablation balloon for occluding a cavity of a subject during the procedure, the device comprising:
a processor circuit comprising:
an input for receiving data representing one or more electrical signals measured using one or more of a plurality of electrodes disposed on an elongate tip member of an electrophysiology catheter when one or more of the plurality of electrodes are positioned distally of the ablation balloon in the anatomical cavity wherein the electrical signals are responsive to local dielectric properties within the anatomical cavity and were measured responsive to injection of a dielectric medium into the anatomical cavity;
a data processor communicatively coupled to the input and configured to:
process the obtained data and identify a change of at least one characteristic of the one or more electrical signals where the at least one change is responsive to the injection of the dielectric medium;
determine from the identified at least one change occlusion information relating to the occlusion of the anatomical cavity by the ablation balloon; and
optionally, an output, communicatively coupled to the data processor, for outputting output data comprising the occlusion information.

2. The device of claim 1, wherein the at least one change of characteristic comprises one or more of:
the amplitude of a signal spike;
the time decay of a signal spike; and
a comparison of the signal amplitude before and after a signal spike.

3. The device of claim 1, wherein the data represent a plurality of electrical signals each one measured using one of the plurality of electrodes and wherein the data processor is configured to identify the at least one change of the characteristic for each of the plurality of signals.

4. The device of claim 1 wherein the data processor is further configured to determine, based on the change of the at least one characteristics changes in local dielectric properties within the cavity.

5. The device of claim 1, wherein the electrical signals further comprise baseline electrical signals measured when the anatomical cavity is not occluded by the ablation balloon and wherein the data processor is configured to determine a degree of occlusion using a model based on a comparison of the baseline signal to a signal obtained after occlusion.

6. The device of claim 1, wherein the processor circuit is configured to:
communicate with the one or more of a plurality of electrodes disposed on the elongate tip member of the electrophysiology catheter, and
control the one or more of the plurality of electrodes to provide a local electric field and measure the electrical signals.

7. The device of claim 1, wherein the processor circuit is further configured to generate the occlusion information to comprise a visualization of at least part to of the occlusion information indicating whether the ablation balloon at least partially occludes the anatomical cavity corresponding to, near, or at a location of each of the one or more of the plurality of electrodes.

8. The device of claim 7 wherein the processor circuit is further configured to:
receive, at the input, data representative of a map of at least part of the anatomical cavity relevant to the procedure; and
generate the map;
generate a visualization of the occlusion information; and
generate output data comprising the map and the visualization.

9. The device of claim 1, wherein the processor circuit is configured to:
communicate with the one or more of the plurality of electrodes and with a plurality of external body patch electrodes for positioning on a subject to apply an electrical field to at least the anatomical cavity using body patch electrical signals, wherein the processor circuit is configured to:
control the external body patch electrodes to provide the electrical field;
control the one or more of the plurality of electrodes to detect distortions in the generated electrical field and generate map data comprising the detected distortions;
generate, based on the detected distortions, a map of the anatomical cavity relevant to the procedure;

10. A system for assisting a balloon ablation therapy procedure using an ablation balloon for occluding a cavity of a subject during the procedure, comprising:
a device as claimed in claim 1 wherein the processor circuit comprises an output, communicatively coupled to the data processor; and
a user interface communicatively coupled to the processor circuit at least via the output and configured to provide an indication of the occlusion information to a user.

11. The system of claim 10, comprising
a controller for communicatively coupling to the one or more of the plurality of electrodes and to the plurality of body patch electrodes, and communicatively coupled to the processing circuit, the controller being configured to supply any electrical signals for any electrodes upon control of any of the electrodes by the processing circuit;

12. The system of claim 10, comprising an injection system for injecting the medium, and optionally a vessel comprising the medium.

13. The system of claim 10 comprising:
the ablation balloon;
the electrophysiology catheter.

14. A method for assisting a balloon ablation therapy procedure using an electrophysiology catheter and ablation balloon for occluding a cavity of a subject during the procedure during which procedure data are generated that represent one or more electrical signals measured using one or more of a plurality of electrodes disposed on an elongate tip member of the electrophysiology catheter when one or more of the plurality of electrodes are positioned distally of the ablation balloon in the anatomical cavity and wherein the electrical signals are responsive to local dielectric properties within the anatomical cavity and were measured responsive to injection of a dielectric medium into the anatomical cavity, the method comprising:
receiving, at an input of a processor circuit, the data;
processing, by a data processor communicatively coupled to the input, the received data and identify a change of at least one characteristic of the one or more electrical signals where the at least one change is responsive to the injection of the dielectric medium;
determining, by the data processor, from the identified at least one change occlusion information relating to the occlusion of the anatomical cavity by the ablation balloon; and
optionally, provide, using a user interface communicatively coupled to an output of the processor circuit and communicatively coupled to the data processor, the occlusion information to a user.

15. The method of claim 14, wherein the at least one change of characteristic comprises one or more of:
the amplitude of a signal spike;
the time decay of a signal spike; and
a comparison of the signal amplitude before and after a signal spike.

16. The method of claim 14, wherein the electrical signals further comprise baseline electrical signals measured when the anatomical cavity is not occluded by the ablation balloon and wherein the data processor is configured to determine a degree of occlusion using a model based on a comparison of the baseline signal to a signal obtained after occlusion.

17. The method of claim 14, comprising controlling the one or more of the plurality of electrodes to measure the electrical signals.

18. The method of claim 14 comprising:
receiving, at the input, data representative of a map of at least part of the anatomical cavity relevant to the procedure; and
generating the map;
generating a visualization of the occlusion information; and
generating output data comprising the map and the visualization.

19. A computer program product comprising instructions for implementing the method when said program is run on a processor circuit and/or data processor of the device of claim 1.

20. A computer readable medium comprising the computer program product of claim 19.
